(19) United States
(12) Patent Application Publication
(10) Pub. No.: US 2003/0219770 A1
(43) Pub. Date: Nov. 27, 2003

(54) Methods and systems of nucleic acid sequencing

(76) Inventors: James R. Eshleman, Lutherville, MD (US); Kathleen M. Murphy, Baltimore, MD (US)

Correspondence Address: EDWARDS & ANGELL, LLP, P.O. BOX 9169, BOSTON, MA, 02209, US

(21) Appl. No.: 10/291,205

(22) Filed: Nov. 8, 2002

Related U.S. Application Data
(60) Provisional application No. 60/348,202, filed on Nov. 8, 2001.
(60) Provisional application No. 60/332,317, filed on Nov. 9, 2001.
(60) Provisional application No. 60/361,125, filed on Mar. 1, 2002.

(51) Int. Cl. C12Q 1/68; G06F 19/00; G01N 33/48; G01N 33/50

(52) U.S. Cl. 435/6; 702/20

(57) ABSTRACT

Methods for the simultaneous sequencing of multiple nucleic acid molecules are provided. Preferred methods include simultaneous single-direction sequencing of multiple genes or forward and reverse sequencing from a single gene, within a single reaction vessel. Additional methods of the invention include combined amplification and sequencing of nucleic acids, from a variety of sources, within a single reaction and wherein nucleic acid products also can be simultaneously analyzed, and where the reaction can be either bidirectional or long unidirectional. Additional methods encompass combined amplification and sequencing of multiple nucleic acid molecules simultaneously.

This application claims the benefit of U.S. Provisional Application No. 60/348,202 filed, Nov. 8, 2001, U.S. Provisional Application No. 60/332,317 filed, Nov. 9, 2001 and U.S. Provisional Application No. 60/361,125 filed Mar. 1, 2002, all of which applications are incorporated herein by reference in their entirety.

### BACKGROUND OF THE INVENTION

1. Field of the Invention

The present invention is generally directed to methods for the simultaneous sequencing of multiple nucleic acid molecules derived from a variety of sources, without the need to perform each reaction separately. More specifically, the present invention provides methods for simultaneous single-direction sequencing of multiple genes or forward and reverse sequencing from a single gene, within a single reaction vessel. The present invention also provides for methods wherein amplification and sequencing of nucleic acids, from a variety of sources, is performed in a single reaction. Nucleic acid products are also simultaneously analyzed.

2. Background

DNA sequencing (1) has been the standard against which other types of DNA testing is compared. Major advances in DNA sequencing include the development of “automated” sequencers (2), discovery of fluorescent terminator chemistry (3) and cycle sequencing. These developments have made sequencing easier to perform and therefore more widely used. Currently, sequencing is used to identify microbial drug resistance mutations (4), cancer predisposition mutations (5), and genetic diseases (6). With the cloning and sequencing of the human genome (7, 8) and the new era of molecular medicine, one can only expect the use of DNA sequencing to increase.

In DNA sequencing by the enzymatic chain termination method according to Sanger one starts with a nucleic acid template from which many labeled nucleic acid fragments of various length are produced by an enzymatic extension and termination reaction in which a synthetic oligonucleotide primer is extended and terminated with the aid of polymerase and a mixture of deoxyribonucleoside triphosphates (dNTP) and chain termination molecules, in particular dideoxyribonucleoside triphosphates (ddNTP).

In this method a mixture of the deoxyribonucleoside triphosphates (dNTPs) and one dideoxyribonucleoside triphosphate (ddNTP) is used in each of four reaction mixtures. In this manner a statistical incorporation of the chain termination molecules into the growing nucleic acid chains is achieved and after incorporation of a chain termination molecule the DNA chain cannot be extended further due to the absence of a free 3′-OH group. Hence, numerous DNA fragments of various length are formed which, from a statistical point of view, contain a chain termination molecule at each potential incorporation site and end at this position. These four reaction mixtures which each contain fragments ending at a base due to the incorporation of chain termination molecules are separated according to their length for example by polyacrylamide gel electrophoresis and usually in four different lanes and the sequence is determined by means of the labeling of these nucleic acid fragments.

Presently, DNA sequencing is carried out with automated systems in which usually a non-radioactive label, in particular a fluorescent label, is used (L. M. Smith et al, Nature 321 (1986), 674-679; W. Ansorge et al, J. Biochem. Biophys. Meth. 13 (1986), 315-323). In these automated systems the nucleotide sequence is read directly during the separation of the labeled fragments and entered directly into a computer.

In the automated methods for sequencing nucleic acids non-radioactive labeling groups can either be introduced by means of labeled primer molecules, labeled chain termination molecules or as an internal label via labeled dNTP. In all these known labeling methods the sequencing reactions are in each case carried out individually in a reaction vessel so that always only one single sequence is obtained with a sequencing reaction.

Despite advances in sequencing technology, significant limitations remain. First, most applications require polymerase chain reaction (PCR) amplification of the target sequence, and purification of the product prior to sequencing. Second, standard Sanger sequencing reactions are carried out with a single primer and therefore yield only a single sequence. These limitations have hindered the widespread application of DNA sequencing in clinical and research settings.

Polymerase chain reaction (PCR) amplification of genes has been the cornerstone for sequencing. However, PCR, especially multiplex PCR, have limitations, especially when genes from a variety of sources are present in the sample. For example, allele-specific PCR products generally have the same size, and an assay result is scored by the presence or absence of the product band(s) in the gel lane associated with each reaction tube. Gibbs et al., Nucleic Acids Res., 17:2437-2448 (1989). This approach requires splitting the test sample among multiple reaction tubes with different primer combinations, multiplying assay cost. PCR has also discriminated alleles by attaching different fluorescent dyes to competing allelic primers in a single reaction tube (F. F. Chehab, et al., Proc. Natl. Acad. Sci. USA, 86:9178-9182 (1989)), but this route to multiplex analysis is limited in scale by the relatively few dyes which can be spectrally resolved in an economical manner with existing instrumentation and dye chemistry. The incorporation of bases modified with bulky side chains can be used to differentiate allelic PCR products by their electrophoretic mobility, but this method is limited by the successful incorporation of these modified bases by polymerase, and by the ability of electrophoresis to resolve relatively large PCR products which differ in size by only one of these groups. Livak et al., Nucleic Acids Res., 20:4831-4837 (1989). Each PCR product is used to look for only a single mutation, making multiplexing difficult.

Ligation of allele-specific probes generally has used solid-phase capture (U. Landegren et al., Science, 241:1077-1080 (1988); Nickerson et al., Proc. Natl. Acad. Sci. USA, 87:8923-8927 (1990)) or size-dependent separation (D. Y. Wu, et al., Genomics, 4:560-569 (1989) and F. Barany, Proc. Natl. Acad. Sci., 88:189-193 (1991)) to resolve the allelic signals, the latter method being limited in multiplex scale by the narrow size range of ligation probes. Further, in a multiplex format, the ligase detection reaction alone cannot make enough product to detect and quantify small amounts of target sequences. The gap ligase chain reaction process requires an additional step-polymerase extension. The use of probes with distinctive ratios of charge/translational frictional drag for a more complex multiplex will either require longer electrophoresis times or the use of an alternate form of detection.

### SUMMARY OF THE INVENTION

The present invention provides for novel sequencing strategies which directly address the limitations in sequencing methods. Specifically, the invention provides for engineered sequencing reactions to permit simultaneous sequencing of multiple polymerase chain reaction (PCR) products in a single sequencing reaction and simultaneous analysis without the need to separate the products prior to analysis. In another sequencing strategy, the invention provides for combined PCR and sequencing in a single reaction and simultaneous analysis.

In particular, novel sequencing reactions were engineered to permit simultaneous sequencing of multiple polymerase chain reaction (PCR) products in a single lane. Under normal conditions, multiple sequencing reactions run simultaneously would be superimposed on each other because the sequencing products overlap in size. This sequencing strategy prevents this because of two principles: sequencing products stop when the end of a PCR product is reached, and long oligonucleotide primers can be used to prevent short sequencing products.

In another embodiment, sequencing conditions and primer modifications to permit combined simultaneous sequencing in a single reaction are provided for. In particular, the method provides for uni-directional and bi-directional (combined forward and reverse sequencing), with or without prior amplification. For bi-directional sequencing, the preferred modifications include introduction of an abasic region between the short region of the primer that is homologous to the DNA gene template and the long region of non-templated nucleotides tailed on the 5′ end. This modification prevents forward primer extension products from extending down the reverse primer and its products.

In another preferred embodiment, an abasic region is introduced into the primer between the short region homologous to the DNA template and the long non-templated thymidines.

In another preferred embodiment the reverse PCR primer is functionally removed to increase the number of genes that can be simultaneously sequenced. Removal of redundant reverse PCR primers from PCR products prior to sequencing allows for more sequencing reactions to be performed. The preferred method for removing the reverse PCR primer is Uracil N-DNA glycosylase.

In another preferred embodiment the method of PCR and simultaneous nucleic acid sequencing is combined in a single reaction in the same reaction vessel. In particular, nucleic acid sequence of interest is amplified using the polymerase chain reaction, which is obtained initially by increasing the free nucleotide concentration as compared to the nucleotide concentrations used in standard sequencing methods. During the polymerase chain reaction, the nucleotide concentration is depleted by the amplification process, thereby raising the relative concentration of di-deoxynucleotides and favoring sequencing rather than amplification.

In a preferred embodiment the PCR and simultaneous sequencing provides for bi-directional sequencing in a single reaction, within the same reaction vessel.

In another preferred embodiment, PCR and simultaneous sequencing long uni-directional sequencing with PCR are performed in a single reaction within the same reaction vessel. Most preferably, this is achieved using unmodified oligonucleotide primers in unequal molar ratios, for example, the ratio of forward: reverse primers can be 5:1, 10:1, 20:1, 1:5, 1:10, 1:20, although other ratios could be used. Alternatively, this is achieved by altering the position of the forward primer relative to the PCR product and by using a longer modified reverse primer.

Preferred modified primers, include modifications which are not restricted to, abasic regions; a string of non-homologous thymidines; immobilization of the reverse primer or slowing the migration of a primer in a gel or column by using branched DNA or biotinylated primers reacted with avidin or avidin conjugated beads; cleavage of the sugar backbone; addition of blocking groups and the like.

In separate embodiments, the reporter molecules useful within the methods of the present invention include such molecules as biotin, digoxigenin, hapten and mass tags or any combination of these.

In other embodiments, the present invention employs selected nucleotides, or functionally equivalent structures, to provide linkages for detectors and reporter binding molecules of different kinds, such linkages utilizing different deoxynucleoside phosphates as well as abasic nucleotides and nucleosides selectively structured and configured so as to provide an advantage in detecting the resulting rolling circle products. Reporter molecules may also include enzymes, fluorophores and various conjugates.

In another preferred embodiment, the PCR and simultaneous sequencing reaction, includes but is not limited to any amplification procedures such as for example, polymerase chain reaction (PCR), multiplex PCR, Rolling Circle PCR (RCA), long chain polymerase reaction, ligase chain reaction, reverse transcriptase PCR (RT-PCR), differential display PCR, self-sustained sequence replication (3SR), nucleic acid sequence based amplification (NASBA), strand displacement amplification (SDA), and amplification with Qβ-replicase (Birkenmeyer and Mushahwar, J. Virological Methods, 35:117-126 (1991); Landegren, Trends Genetics, 9:199-202 (1993)), linear rolling circle amplification (LRCA) uses a primer annealed to the circular target DNA molecule and DNA polymerase is added, exponential RCA (ERCA) with additional primers that anneal to LRCA product strand.

These novel sequencing strategies have the advantages of being easily implemented in any lab currently performing sequencing, as the strategies are not labor intensive as compared to present methods; multiple gene sequencing or forward and reverse sequencing is conducted in a single reaction vessel; the products of the sequencing reaction are analyzed in a single lane in a gel or capillary; polymerase chain reaction (PCR) and sequencing are conducted in the same reaction vessel without the requirement to remove residual free PCR primers and nucleotides; no additional dyes, special equipment or strand separation steps are required; labor and costs are significantly decreased compared to the present state of the art.

The invention also provides kits useful for conducting methods and assay of the invention. Preferred kits comprise suitable primers as disclosed herein, include thymidine primers and extended primers.

Other aspects of the invention are disclosed infra.

### BRIEF DESCRIPTION OF THE DRAWINGS

FIG. 1 (includes FIGS. 1A-1E) is a schematic representation using simultaneous sequencing (“SimulSeq”) of three genes.

FIG. 1A depicts a schematic of the experimental design.

FIGS. 1B-1E provide the results of simultaneous sequencing of PCR products from methylenetetrahydrofolate reductase (MTHFR), prothrombin (PROT), and Factor V (FV) genes demonstrating (B) Factor V Leiden, (C) prothrombin, and (D) MTHFR heterozygotes, respectively.

FIG. 2 (includes FIGS. 2A-2C) illustrates the results obtained with bi-directional simultaneous sequencing.

FIG. 2A is a schematic representation of the experimental design of simultaneous forward and reverse sequencing.

FIG. 2B illustrates the results obtained using simultaneous forward and reverse sequencing of homozygous wild type (WT/WT) and heterozygous Leiden mutant (WT/L) individuals.

FIG. 2C illustrates the results of a conventional RFLP assay for Factor V Leiden mutation using a non-denaturing 10% polyacrylamide gel electrophoresis (PAGE) of PCR products following restriction digest with MnlI and ethidium bromide staining.

FIG. 3 (includes FIGS. 3A-3C) is an illustrative example using combined amplification and sequencing (“AmpliSeq”).

FIG. 3A is a schematic illustration of the anticipated PCR product generated during combined amplification/sequencing.

FIG. 3B illustrates the results obtained using bi-directional combined amplification/sequencing of Factor V wildtype homozygote.

FIG. 3C illustrates the results obtained using unidirectional amplification/sequencing of Factor V wildtype homozygote.

FIG. 4 is a schematic illustrative representation of uni-directional sequencing using SimulSeq.

FIG. 5 is a schematic illustrative representation of bi-directional sequencing using SimulSeq.

FIG. 6 is a schematic illustrative representation of simultaneous PCR and sequencing within the same reaction vessel, using the method, herein referred to as AmpliSeq.

FIG. 7 is a schematic of a method of the invention providing long uni-directional sequencing, using the modified reverse primer strategy.

FIG. 8 demonstrates results providing long unidirectional sequencing of two separate genes, using the unmodified normal primers at non-equal molar ratio approach.

FIG. 9 is a schematic of a method of the invention providing long unidirectional sequencing, using the unmodified normal primers at non-equal molar ratio approach.

FIG. 10 demonstrates results showing combined SimulSeq and AmpliSeq (in a single tube, combined amplification and sequencing of two products simultaneously).

FIG. 11 is a schematic of a method of the invention demonstrating combined SimulSeq and AmpliSeq (in a single tube, combined amplification and sequencing of two products simultaneously).

### DETAILED DESCRIPTION OF THE INVENTION

The present invention is generally directed to methods for the simultaneous sequencing of multiple nucleic acid molecules derived from a variety of sources, without the need to perform each reaction separately. In addition, amplification of nucleic acids by polymerase chain reaction and subsequent sequencing of the products generated, are sequenced in the same reaction vessel without the need for separating and purifying the products, as is the usual custom, prior to carrying out the sequencing of the PCR products. The products, thus generated, are analyzed as if the source of genetic material was derived from a single sample, thereby circumventing any need to separate samples into multiple reaction vessels prior to analysis.

In one aspect, the invention allows for, either simultaneous single-direction sequencing of multiple genes or simultaneous bidirectional sequencing from a single gene following PCR. This method is often referred to herein as “SimulSeq”.

This method has several advantages over previously described methods of simultaneous sequencing which require significant deviations from standard sequencing protocols. For example, one method employs two fluorescently labeled primers, and specialized detection equipment and software to “sort” the sequence data (14-16), while another method requires strand separation following the sequencing reaction and separate sequence analysis (17). See Ref 17: van den Boom, D., et al. (1998) Anal Biochem 256, 127-9.

SimulSeq can be applied to a plethora of gene analysis methods, for example, detection of mutation sites, detection of genetic polymorphism, clinical diagnostics, forensics, detection of single nucleotide polymorphisms (SNP), large scale genetic testing, analysis of bioterrorism organisms, and drug resistance testing, and the like.

SimulSeq reactions can be designed to yield many short sequences, fewer long sequences, or a combination of short and long sequences. Thus SimulSeq can be adapted for many different types of simultaneous sequencing applications.

In another aspect of the invention, PCR and cycle sequencing are combined in a single reaction that yields both forward and reverse sequence data. In accordance with the invention, PCR and cycle sequencing can be combined in a strategy to produce long unidirectional sequencing. This method will be referred to herein as “AmpliSeq”. No other methods, have up until now, effectively combining PCR and sequencing in a single reaction. Previously attempts require that the samples be partitioned after several cycles of amplification so that radioactively labeled primers and di-deoxynucleotides can be added to 8 individual reactions (18, 19). See Ref 18: Ruano, G., et al (1991) Proc Natl Acad Sci USA 88, 2815-9. Although AmpliSeq and SimulSeq can require attention to primer design, they can require no additional steps, sample manipulations, or reagents such that any lab currently performing DNA sequencing reactions can perform either of these techniques. These techniques can significantly reduce the cost, time, and labor of nucleic acid sequencing, making direct sequencing a competitive alternative to other mutation detection methods and are ideally suited for a variety of clinical and research applications, such as SNP panels, large scale genetic testing, analysis of bioterrorism organisms, and drug resistance testing.

SimulSeq and AmpliSeq also can provide a major improvement over current technology in the area of diagnostic sequencing. An ever widening array of disorders, susceptibilities to disorders, prognoses of disease conditions, and the like, have been correlated with the presence of particular DNA sequences, or the degree of variation (or mutation) in DNA sequences, at one or more genetic loci. Examples of such phenomena include human leukocyte antigen (HLA) typing, cystic fibrosis, tumor progression and heterogeneity, p53 proto-oncogene mutations, ras proto-oncogene mutations, and the like, e.g. Gyllensten et al, PCR Methods and Applications, 1: 91-98 (1991); Santamaria et al, International application PCT/US92/01675; Tsui et al, International application PCT/CA90/00267; and the like. A difficulty in determining DNA sequences associated with such conditions to obtain diagnostic or prognostic information is the frequent presence of multiple subpopulations of DNA, e.g. allelic variants, multiple mutant forms, and the like. Distinguishing the presence and identity of multiple sequences with current sequencing technology is virtually impossible, without additional work to isolate and perhaps clone the separate species of DNA.

SimulSeq and AmpliSeq also can fulfill the growing need (e.g., in the field of genetic screening) for methods useful in detecting the presence or absence of each of a large number of sequences in a target polynucleotide. For example, as many as 400 different mutations have been associated with cystic fibrosis. In screening for genetic predisposition to this disease, it is optimal to test all of the possible different gene sequence mutations in the subject's genomic DNA, in order to make a positive identification of “cystic fibrosis”. It would be ideal to test for the presence or absence of all of the possible mutation sites in a single assay. However, prior art methods are not readily adaptable for use in detecting multiple selected sequences in a convenient, automated single-assay format.

In one method aspect, the invention provides approaches for substantially simultaneously sequencing multiple DNA oligonucleotides, which may be pooled from a variety of sources, in a single reaction using a single reaction vessel. Such methods generally include providing a plurality of DNA oligonucleotides; providing a plurality of primers; contacting or annealing of the primers to target sequences of the oligonucleotides; sequencing the DNA oligonucleotides using the primers to obtain a pool of sequence data; and analyzing the sequence data without the need to separate the pool of sequence data prior to analysis. Preferably, the pool of sequence data is analyzed substantially simultaneously (i.e. without separation of components) within a single lane or capillary.

In such methods, a variety of DNA molecules may be employed, including DNA oligonucleotides that are single stranded, DNA oligonucleotides that are double stranded, DNA oligonucleotides that are genes or fragments thereof, with such oligonucleotides being from the same or different genes or gene fragments.

The primers can vary, e.g. in length, modifications and size. Preferred primers may be modified to contain an abasic region. Suitable primers also may comprise non-template 5′ tails of varying lengths. Primers suitably may be specific for different target DNA sequences, or may be specific for the same DNA sequences.

The desired length of the sequence data can be varied according to the design of the primer used. Typically, the shortest desired length of sequence data is at least about one or more bases.

In such methods, the sequencing reaction can be unidirectional or bi-directional. Significantly, in such methods the sequencing reaction does not require the separation of the nucleic acids to be separated into different reaction vessels. Indeed, the sequencing reaction of multiple DNA oligonucleotides, or fragments thereof, is performed in a single step without the need to separate each oligonucleotide into separate reaction vessels. The sequence data can be analyzed without the need to separate each sequence obtained from the sequencing reaction, before analysis of the data.

Preferably, the plurality of target nucleic acid molecules are amplified such as by polymerase chain reactions, prior to sequencing. In such an approach, the reverse polymerase chain reaction primers are suitably removed the amplified products prior to sequencing, such as by an enzymatic treatment, e.g. using uracil N-DNA-glycosylase.

The invention also provides methods for amplifying and substantially simultaneously sequencing a plurality of nucleic acid molecules in a single reaction within a single reaction vessel. In accordance with the invention, the reaction vessel suitably comprises a plurality of target nucleic acid molecules; a plurality of forward and reverse nucleic acid primer molecules, wherein each primer molecule can hybridize to a distinct area of the target nucleic acid molecule.

In accordance with the invention, the target nucleic acid molecules are amplified such as by performing a polymerase chain reaction, suitably wherein deoxyribonucleosides triphosphates are added during the early cycles of the polymerase chain reaction thereby allowing a number of multiple amplification cycles of target nucleic acid molecules, and wherein the number of amplifying cycles are determined by the added concentration of deoxyribonucleosides triphosphates; and as the amplifying cycles consume the added deoxyribonucleosides triphosphates, during which, the concentrations of free deoxyribonucleosides triphosphates decrease thereby raising the concentration of di-deoxyribonucleoside triphosphates. This approach favors a sequencing reaction rather than amplification, i.e. sequencing predominates with respect to amplification at a relative rate of 2:1, more typically 3:1, 4:1, 5:1 or 6:1 or more.

In such methods amplification of target nucleic acid molecules such as via polymerase chain reaction and sequencing of polymerase chain reaction products is performed in a single reaction vessel without the need to process or clean-up the amplified products prior to sequencing. A variety of amplification approaches can be utilized, e.g. a standard polymerase chain reaction, a ligase chain reaction, reverse transcriptase polymerase chain reaction, Rolling Circle polymerase chain reaction, multiplex polymerase chain reaction and the like.

In such methods, the concentration of added free deoxyribonucleosides triphosphates determines the number of amplification cycles. During the amplification cycle, the concentration of di-deoxyribonucleosides triphosphates relative to the deoxyribonucleosides triphosphates increases as the deoxyribonucleosides triphosphates are consumed during the amplification cycle.

Additionally, the relative free concentrations deoxyribonucleosides triphosphates to di-deoxyribonucleosides triphosphates favors a shift from the amplification reaction to a sequencing reaction.

The target nucleic acid molecules suitably can be DNA or RNA. DNA target nucleic acid molecules and RNA target nucleic acid molecules suitably can be single stranded or double stranded. The target nucleic acid molecules suitably can be, for example, genes or fragments thereof, with such oligonucleotides being from the same or different genes or gene fragments; cDNA molecules; non-coding regions of the target molecule; and any combinations, fragments, thereof.

The primers can vary, e.g. in length, modifications and size. Preferred primers may be modified to contain an abasic region. Suitable primers also may comprise a non-template 5′ tails of varying lengths. Primers suitably may be specific for different target DNA sequences, or may be specific for the same DNA sequences.

Suitably in such methods the forward primer is targeted to a different position on the amplified product or alternatively, at the same position, and the reverse primer is of longer length and modified. The modified reverse primer may suitably comprise an abasic region, non-template nucleic acids such as polythymidine tails and is longer in length (e.g. at least 2, 3, 4, 5, 6, 7, 8, 9, 10 or more bases) in relation to the forward primer. Alternatively, the forward primer can be modified.

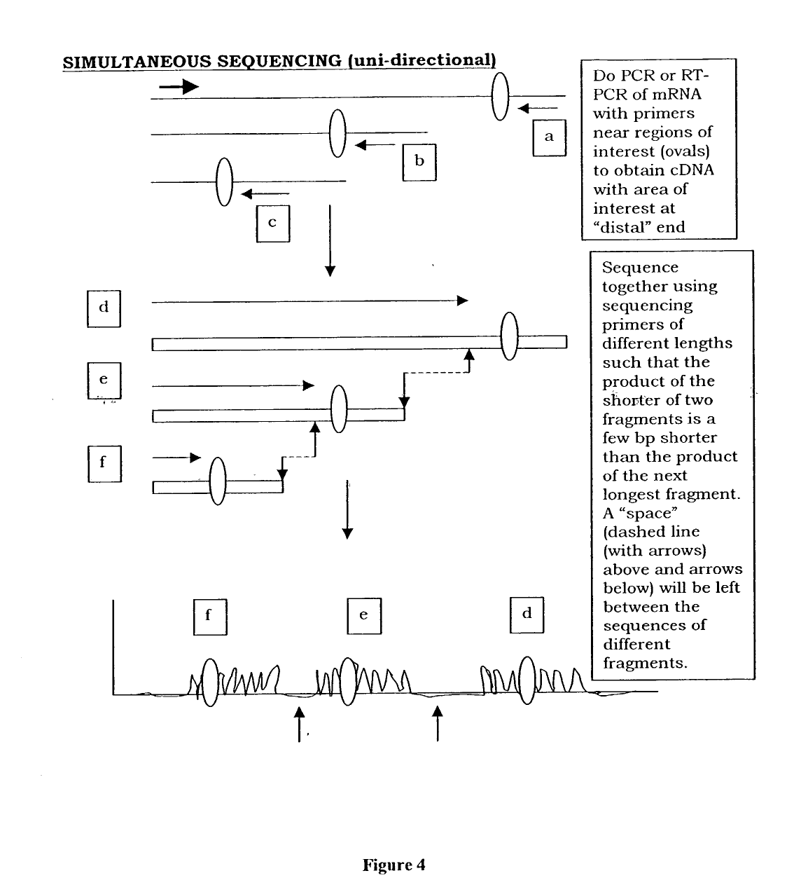

The sequencing reaction suitably can be uni-directional or bi-directional. As used herein, “unidirectional” refers to the sequencing reaction proceeding along one direction of either strand of a nucleic acid molecule. “Bi-directional” is used to refer to the sequencing reaction along proceeding along both strands of a nucleic acid molecule. Illustrative schematic representations of uni-directional and bi-directional sequencing reactions are shown in FIGS. 4 to 7.

Notably, the amplification and sequencing reactions do not require the separation of the nucleic acids into different reaction vessels and are performed in a single step. Additionally, sequencing data obtained from the sequencing reaction is analyzed simultaneously in a single well on a gel or capillary. Sequencing data can be analyzed by immobilizing the reverse primer on a solid support. Preferably, sequencing data is analyzed by using a modified reverse primer such that its migration in the gel or column is slow relative to any other product produced during the amplification and sequencing reactions.

The reverse primer can be modified by biotinylation, blocking group, use of branched primers and the like. Preferably, primers are modified by addition of conjugate molecules that can further increase the binding affinity and hybridization rate of these oligonucleotides to a target. Suitable conjugate molecules may include, cationic amines, intercalating dyes, antibiotics, proteins, peptide fragments, and metal ion complexes. The primers are modified to increase avidity of binding and hybridization rates between a primer and its target nucleic acid, e.g. by 2′ modifications to a ribofuranosyl ring of a primer, particularly a 2′-O-methyl substitution.

As used herein, the term “abasic” refers to a base that is absent from a position in nucleotide sequence.

As an illustrative example, to demonstrate simultaneous sequencing of multiple DNA targets, three genes were simultaneously sequenced. This example is merely for illustrative purposes and is not meant to limit or construe the invention in any way.

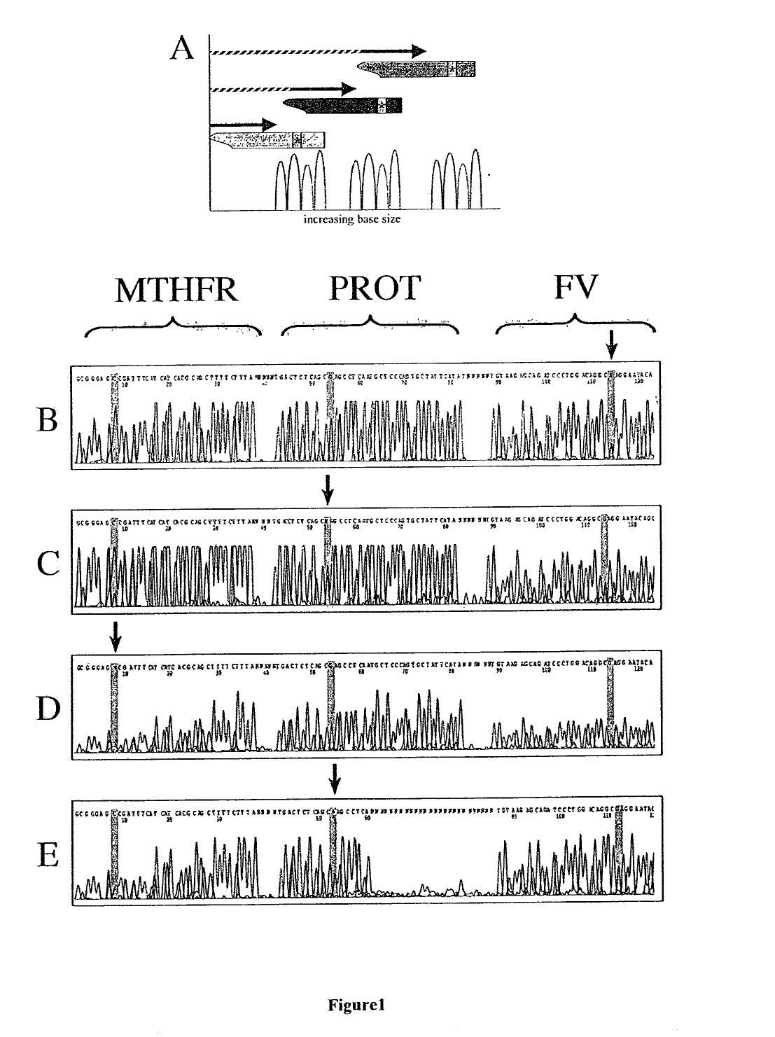

Factor V Leiden (Arg506Gln) (10), prothrombin (G20210A) (11), and the methylenetetrahydrofolate reductase (MTHFR, Ala223Val) (12) mutations each result in an increased risk of thrombosis, and mutations in combination appear to have a synergistic effect on thrombosis risk (13). The experimental strategy is depicted in FIG. IA. PCR of each gene was designed with the known mutation site near the distal end of the PCR strand to be sequenced. This design is required in SimulSeq reactions so that sequencing products terminate shortly after the site of interest, allowing sequencing products generated from other targets to be detected downstream. PCR amplification of each gene was performed in separate PCR reactions using the primers listed in Table 1. The 3 PCR products were mixed at equal concentrations and simultaneously sequenced using a mixture of 3 forward sequencing primers (Table 1), one for each gene, in a single tube, typically using BigDye 2.0 or 3.0 terminator chemistry (Applied Biosystems), template concentrations, primer concentrations, and cycling conditions per manufacturer (95° C. ×10 secs, 50° C.×15 secs, 60° C.×4 mins, ×35 cycles). The results of simultaneous sequencing of the three genes are shown in FIGS. 1B-E. During simultaneous sequencing, the 22 base MTHFR sequencing primer extends up to 42 bases to the end of the PCR product such that the largest MTHFR sequence product was 64 bases in length. A 69 base prothrombin sequencing primer was designed with 24 complementary bases tailed with an additional 45 thymidines on the 5′ end of the primer. This design creates a 6 base gap in sequencing products between the final MTHFR sequencing product (64 bases) and the beginning of prothrombin sequencing products (70 bases) making it easy to distinguish the two. Prothrombin sequence extends up to 39 bases to the end of the PCR product such that the final prothrombin sequence product is 108 bases. A 113 base factor V sequencing primer was designed with 23 complimentary bases tailed with an additional 90 thymidines on the 5′ end. This creates a gap between the final prothrombin sequencing product and factor V sequencing products, which begin at 114 bases and continue up to 183 bases to the end of the PCR product. FIGS. 1B-D demonstrates simultaneous sequencing of the three prothrombotic genes on each of three patients heterozygous for factor V Leiden (FIG. 1B), prothrombin (FIG. 1C), or MTHFR (FIG. 1D) mutations.

An illustrative example of how additional modifications to this method can be used to obtain even shorter stretches of sequence in order to permit larger numbers of sequencing reactions to be run simultaneously, is described herewith.

One strategy is to eliminate the majority of sequencing downstream of the mutation site since it reflects the known sequence of the reverse primer, providing no additional information. A prothrombin reverse PCR primer, is designed identical to that used in FIGS. 1B-D except that two thymidines near the 3′ end of the primer are replaced with uracils. After PCR, the prothrombin PCR products are treated with Uracil-N-glycosylase (UNG) and then mixed with MTHFR and factor V PCR products, and simultaneously sequenced with the three sequencing primers as above. UNG treatment creates abasic sites in the prothrombin PCR products, which selectively terminate the prothrombin sequence at the beginning of the reverse primer (FIG. IE).

This technique can be employed to, for example, to simultaneously acquire very short segments, for example, between about 10 to about 50 bases of sequence from many different gene sequences, making SimulSeq a viable method to detect a large panel of mutations or single nucleotide polymorphisms (SNPs). A typically suitable number of bases for sequencing is up to about 20 or 30 bases, more typically up to about 10, 15 or 20 bases.

To illustrate how to obtain both forward and reverse sequence from a single gene product using SimulSeq, the factor V PCR reaction is re-designed such that the mutation site is located near one end of the 145 by PCR product. An illustrative example of primers are: forward primer, 5′-TGCCCACTGCTTAACAAGACCA-3′ (SEQ ID NO:11), and reverse primer, 5′-AAGGTTACTTCAAGGACAAAATAC-3′ (SEQ ID NO:12). A forward sequencing primer, with about 22 bases in length, for example, 5′-AGGACTACTTCTAATCTGGTAAG-3′ (SEQ ID NO:13), is designed to yield up to about 54 bases of sequencing (to the end of the PCR product). An example of a preferred large reverse primer is comprised of about 24 complimentary bases, about 90 non-coding thymidines and about four abasic sites between the coding and non-coding bases (5′-T₉₀-pRpRpRpR-AAGGTTACTTCAAGGACAAAATAC-3′; SEQ ID NO:14).

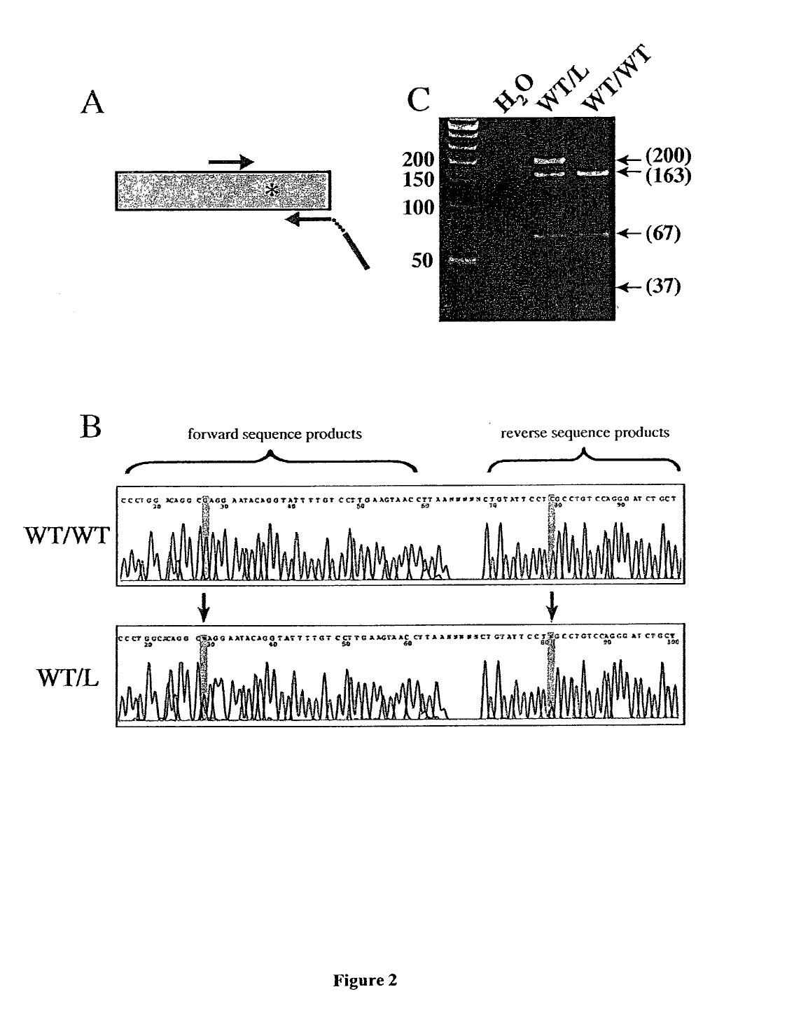

The abasic sites (signified as pR, for phosphate and ribose) are required because products from the reverse primer can serve as templates for the forward primer. Without the reverse primer abasic sites, some forward primer sequencing products terminate within the non-coding thymidine region of the reverse primer and would be superimposed on those generated from the reverse primer. An illustrative experimental design is depicted in FIG. 2A. Bidirectional sequencing for both a factor V wild-type homozygote and Leiden heterozygote is demonstrated in FIG. 2B. As shown, when the forward and reverse primers are used to simultaneously cycle-sequence, there is a short (˜5 base) gap between the end of the forward sequencing products and the beginning of the reverse sequence, making it easy to distinguish the two. The results of simultaneous forward and reverse sequencing correlate with the results of the standard RFLP assay (FIG. 2C).

An illustrative example of combined PCR and cycle sequencing in a single reaction is described as follows. This example is merely for illustrative purposes only and is not meant to construe or limit the invention in any way.

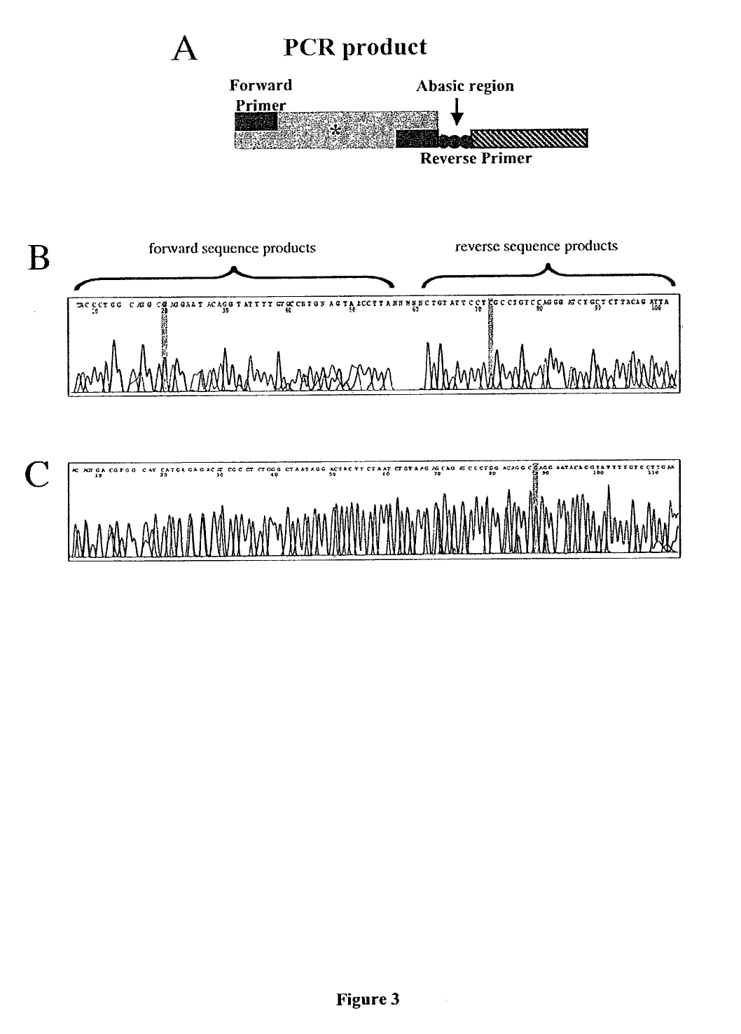

A standard cycle sequencing reaction containing genomic DNA and the factor V forward and reverse primers is used as described above. An anticipated PCR product is diagrammed in FIG. 3A. To support PCR, the reactions are supplemented with additional dNTPs at varying concentrations. Using this approach, early cycles should be dominated by PCR amplification, since the free deoxynucleotide concentration is relatively high, and later cycles by cycle sequencing, because depletion of free deoxynucleotides during PCR increases the relative di-deoxynucleotide concentration. Without deoxynucleotide supplementation, no dissemble sequencing products are identified. With the addition of about 12.5 μM or about 125 μM deoxynucleotides, both forward and reverse sequencing products are generated (FIG. 3B). This illustrative example demonstrates that this method, herein often generally referred to as “AmpliSeq”, supports combined PCR and sequencing in single reactions.

To illustrate yields achieved using modified primer AmpliSeq, input genomic DNA concentrations ranging from about 50 to 500 ng yield approximately equivalent amounts of sequencing products. Primers were added to the reaction at 1 micromolar each, final concentration, and cycle sequenced under the conditions described above.

To illustrate how to generate long unidirectional sequencing (FIG. 3C), using the Factor V example described above, the Factor V AmpliSeq reaction is modified by moving the forward primer further upstream of the Leiden mutation (5′-TGCCCAGTGCTTAACAAGACCA-3′; SEQ ID NO:1), and lengthening the reverse primer tail to about 126 thymidines (5′-T₁₂₆-pRpRpRpR-AAGGTTACTTCAAGGACAAAATAC-3′; SEQ ID NO:10). Therefore, AmpliSeq reactions yield either bidirectional or long unidirectional sequence in combination with PCR amplification.

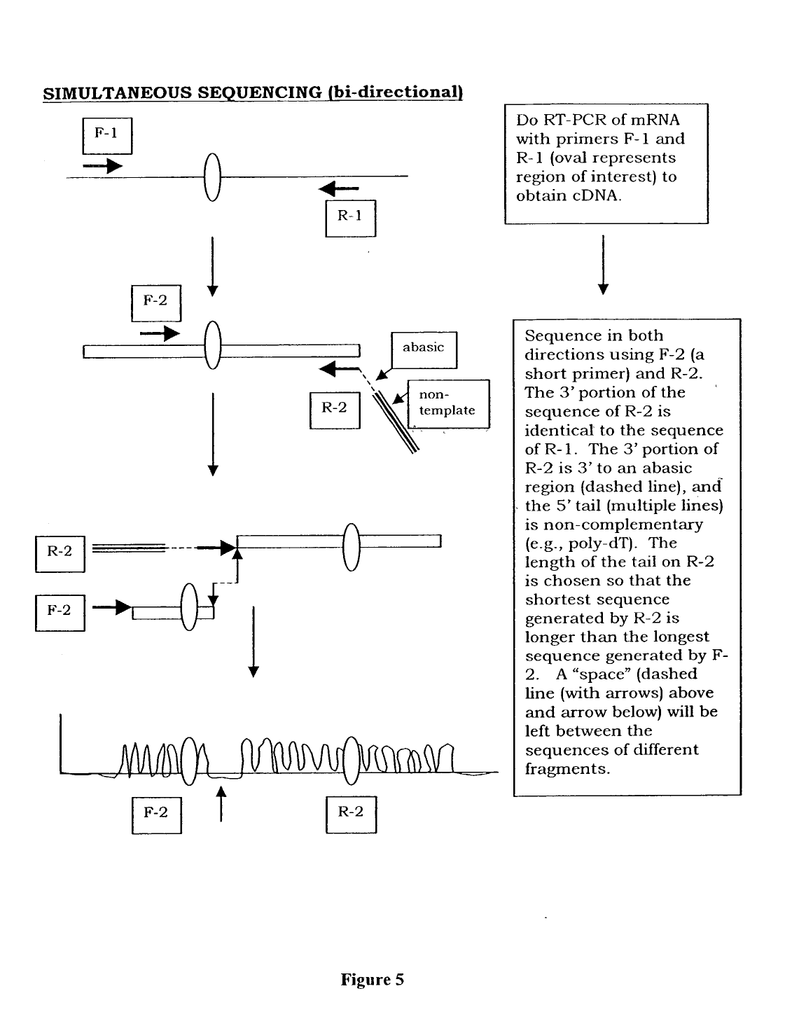

In general SimulSeq and AmpliSeq are illustrated in FIGS. 4 to 6. A general example of using SimulSeq is shown in FIGS. 4 and 5.

FIG. 4 is illustrative of uni-directional sequencing using SimulSeq comprising a the modified reverse primer approach. The basic procedure is performing, for example, RT-PCR of mRNA with primers near regions of interest (ovals) to obtain cDNA with area of interest at “distal” end. Sequence of the product is then performed, using sequencing primers of different lengths, such that the product of the shorter of two fragments is a few bases shorter than the product of the next longest fragment. A “space” (dashed line (with arrows) above and arrows below) is left between the sequences of different fragments. Direct PCR of genomic DNA can similarly be performed.

FIG. 5 is illustrative of bi-directional sequencing using SimulSeq. The basic procedure is performing, for example, RT-PCR of mRNA with primers F-1 and R-1 (oval represents region of interest) to obtain cDNA. Sequencing is then performed in both directions using F-2 (a short primer) and R-2. The 3′ portion of the sequence of R-2 is identical to the sequence of R-1. The 3′ portion of R-2 is 3′ to an abasic region (dashed line), and the 5′ tail (multiple lines) is non-complementary (e.g., poly-dT). The length of the tail on R-2 is chosen so that the shortest sequence generated by R-2 is longer than the longest sequence generated by F-2. A “space” (dashed line (with arrows) above and arrow below) is left between the sequences of different fragments. The abasic region used to stop transcription as there is no template (for bi-directional) resulting in a large and small molecules. Direct PCR of genomic DNA can be similarly be performed.

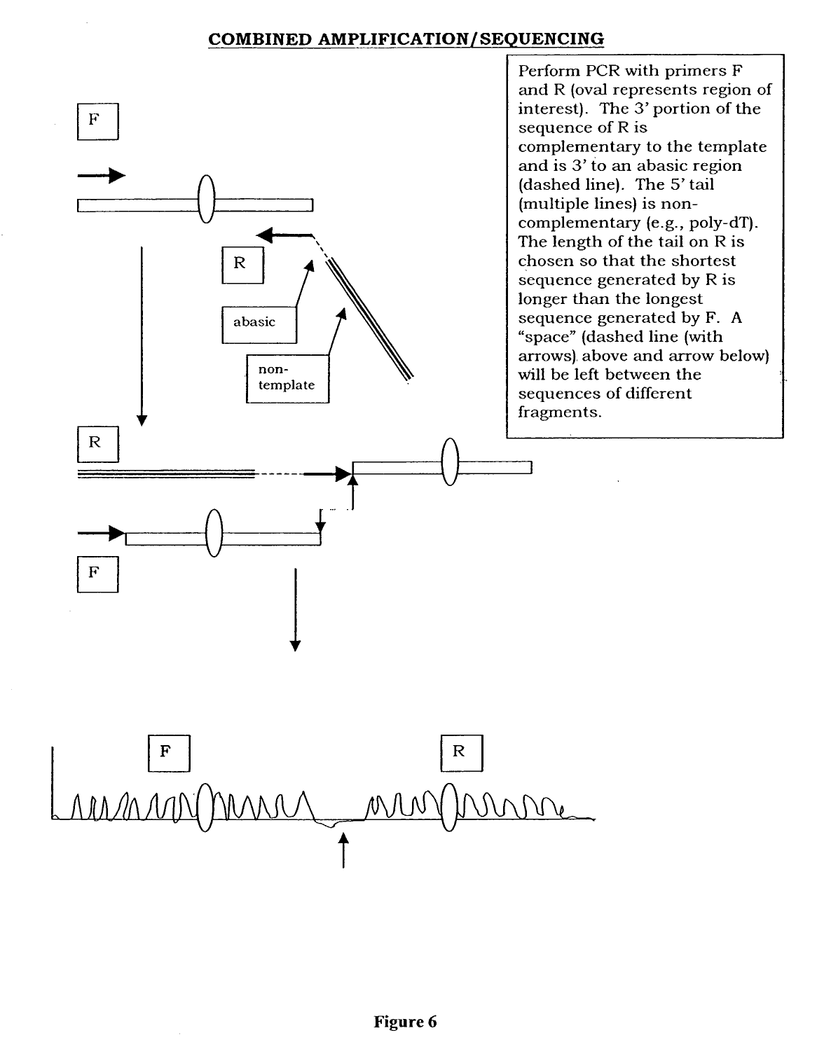

FIG. 6 is illustrative of simultaneous PCR and sequencing within the same reaction vessel, using the method, herein referred to as AmpliSeq. For example, PCR with primers F and R (oval represents region of interest) is first performed. The 3′ portion of the sequence of R is complementary to the template and is 3′ to an abasic region (dashed line). The 5′ tail (multiple lines) is non-complementary (e.g., poly-dT). The length of the tail on R is chosen so that the shortest sequence generated by R is longer than the longest sequence generated by F. A “space” (dashed line (with arrows) above and arrow below) is left between the sequences of different fragments.

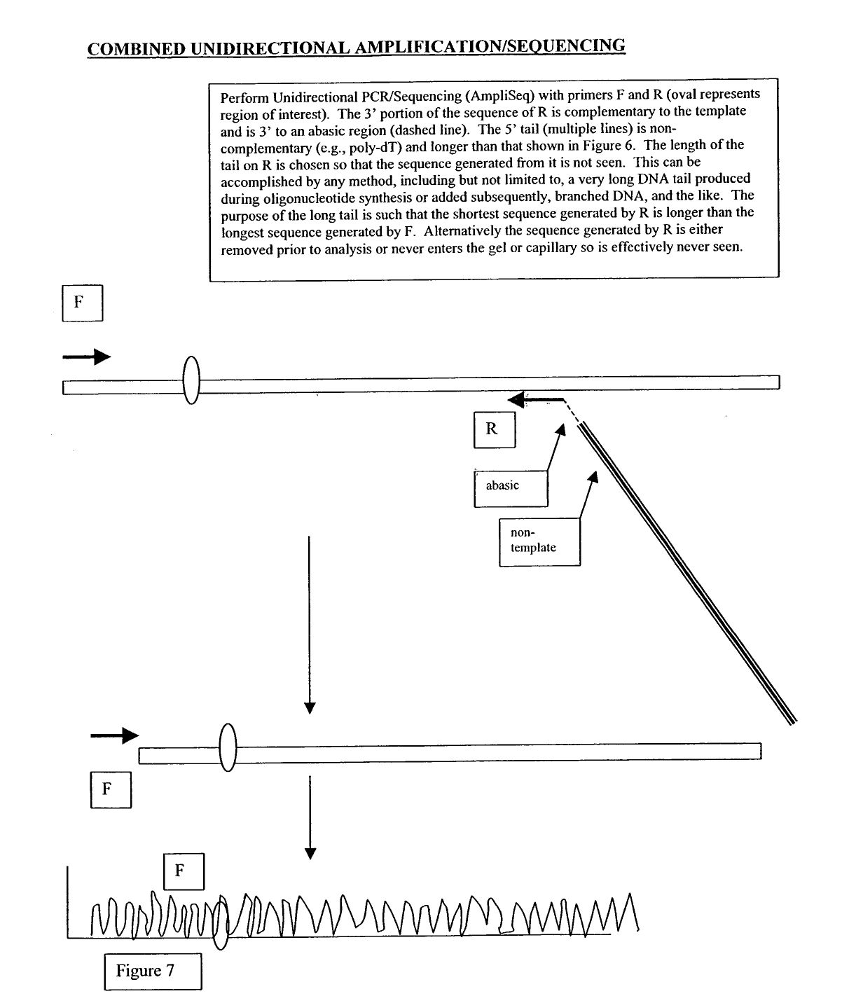

FIG. 7 shows a schematic of performing unidirectional PCR/sequencing (AmpliSeq) with primers F and R (oval represents region of interest). As depicted in FIG. 7, the 3′ portion of the sequence of R is complementary to the template and is 3′ to an abasic region (dashed line). The 5′ tail (multiple lines) is non-complementary (e.g., poly-dT) and longer than that shown in FIG. 6. The length of the tail on R is suitably chosen so that the sequence generated from it is effectively not seen. This can be accomplished by any of a number of methods, e.g. using a very long (e.g. 20, 30, 40, 50, 80, 100, 200, 300 or more bases) produced during oligonucleotide synthesis or added subsequently, branched DNA, and the like. The purpose of the long tail is such that the shortest sequence generated by R is longer (e.g. at least by about 10, 20, 30, 40, 50, 60, 80, 100 or more bases) than the longest sequence generated by F. Alternatively, the sequence generated by R is either removed prior to analysis or never enters in significant amount the gel or capillary so is thereby effectively not seen.

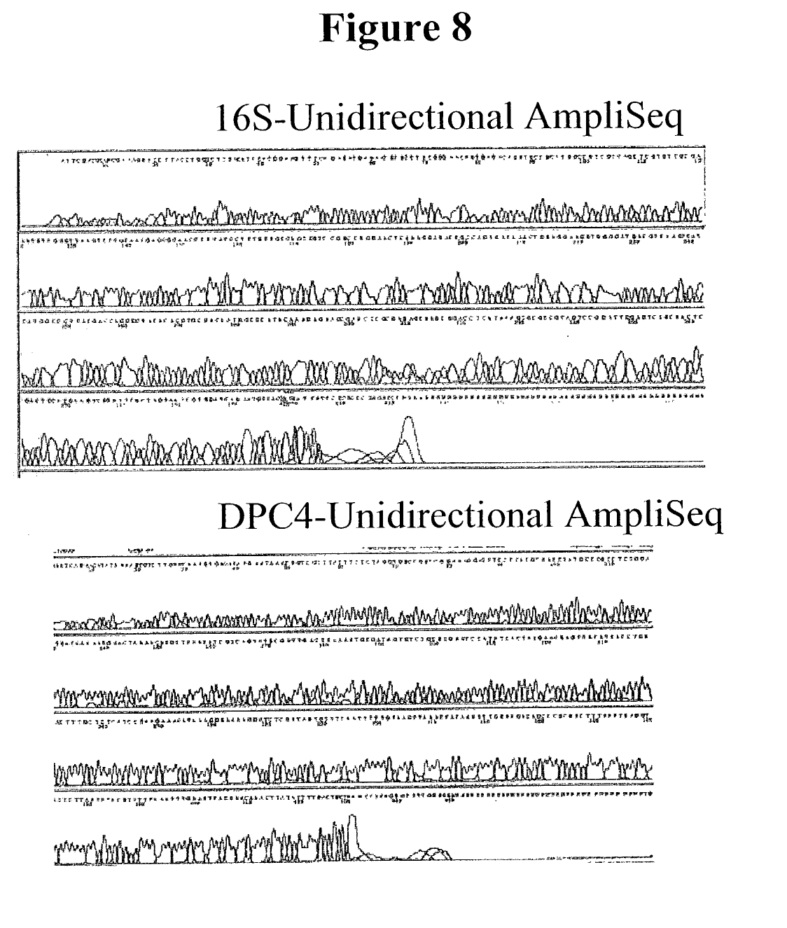

In a preferred embodiment, the altered molar approach is used. Data obtained using the altered molar approach are shown in FIG. 8, which demonstrates use of this approach with unidirectional AmpliSeq. This example is merely for illustrative purposes only and is not meant to construe or limit the invention in any way. The forward primer (5′-CACAAGCGGTGGAGCATGTGG-3′; SEQ ID NO:15) and the reverse primer (5′-AGGCCCGGGAACGTATTCAC-3′; SEQ ID NO:16) were mixed at 5:1 (forward: reverse) molar ratios (final concentration 500 nM forward, 100 nM reverse) with 125 μMolar supplemental dNTPs in Applied Biosystems BigDye 3.0 using 95° C.×15, 50° C.×15, 60° C.×4 mins for 35 cycles conditions and an E. coli DNA target. The results illustrate the number of bases sequenced, approximately greater than 500 bases, though this example is merely for illustrative purposes only and is not meant to construe or limit the invention in any way. Using this method, the full standard-length number of bases is achievable.

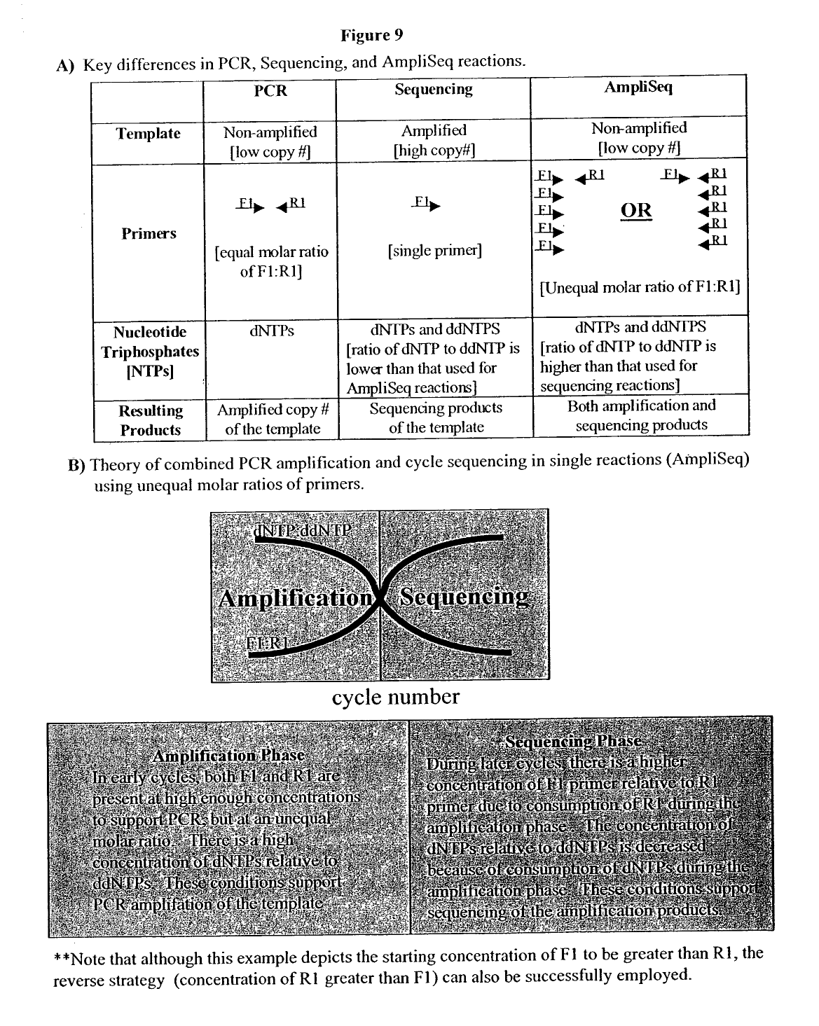

Also shown in FIG. 9, is a tumor-specific mutation in DPC4 (SMAD4). This was generated using the forward, (5′-TAATACTGAGTTGGTAGGATTGTGAG-3′; SEQ ID NO:17) and reverse (5′-CAATACTCGGTTTTAGCAGTC-3′; SEQ ID NO:18) DPC4 primers, under the same conditions as described above.

As used herein, “altered molar approach” refers to the use of non-equal primer molar ratios of forward and reveres primers. For example, to direct a sequencing reaction in the forward direction (i.e. 5′ to 3′ direction) a higher concentration of forward primer is used. An example of a higher concentration would be to use a 15 fold higher concentration of forward primer relative to the concentration of the reverse primer. The concentrations of primers are determined by the methods described in detail in the examples which follow.

As used herein, “non-equal primer molar ratio” refers to the molar ratio of the forward primer as compared to the molar ratio of the reverse primer. For example, the ratio is at least about 2:1 (forward primer: reverse primer) or vice versa depending on the desired direction of the sequencing reaction. The molar ratios, for example, can vary depending on the primers, nucleic acid targets, whether one is using the reaction for detection of small nuclear polymorphisms (SNPs), the direction of the sequencing reaction desired, conditions used, length of primers, whether primers are modified or not and the like. As used herein, the ratios do not have to be in unitary integers, that is (n+1):1, where n=to consecutive numbers, e.g., n=1, 2, 3, 4, 5, and so forth. The non-equal ratios could also be, for example, 15.5:1 or fractions thereof. Concentrations of primers are described in detail in the examples which follow.

FIG. 9 shows a schematic of unidirectional AmpliSeq using the non-equal primer molar ratio approach. FIG. 9A highlights the key differences in the conditions which support standard PCR, standard DNA Sequencing, and AmpliSeq (combined PCR and sequencing together) reactions. It also demonstrates the differences in the products which are produced by each type of reaction. FIG. 9B is a schematic representing the change in relative concentrations of both dNTP:ddNTP and F1:R1, wherein F is the forward primer and R is the reverse primer during AmpliSeq thermocycling. The text below the schematic describes the conditions during both the amplification and sequencing phases of the reaction.

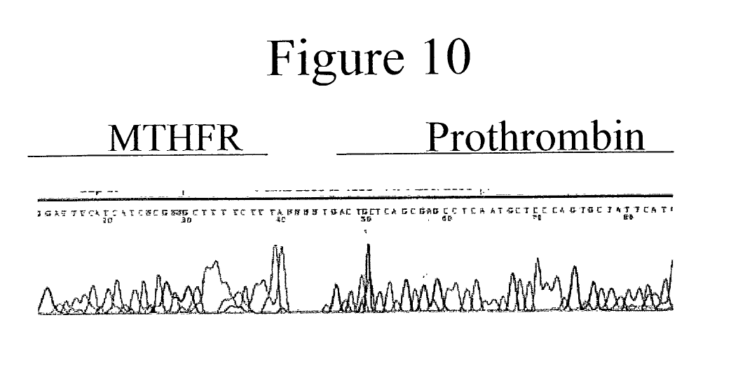

Data is presented in FIG. 10 demonstrating combined PCR and sequencing of two gene products. This example is merely for illustrative purposes only and is not meant to construe or limit the invention in any way. MTHFR and prothrombin primers were mixed where the forward to reverse primer molar ratio was 5:1 (final concentrations, 500 nM and 100 nM) for both primer sets, and added to Applied Biosystems BigDye 3.0 sequencing kit with 125 micromolar supplemental dNTPs and 500 ng human genomic DNA. The primers are from Table 1, where the forward primer is originally used for and listed as the sequencing primer and the reverse primer is that listed under PCR Primers as reverse.

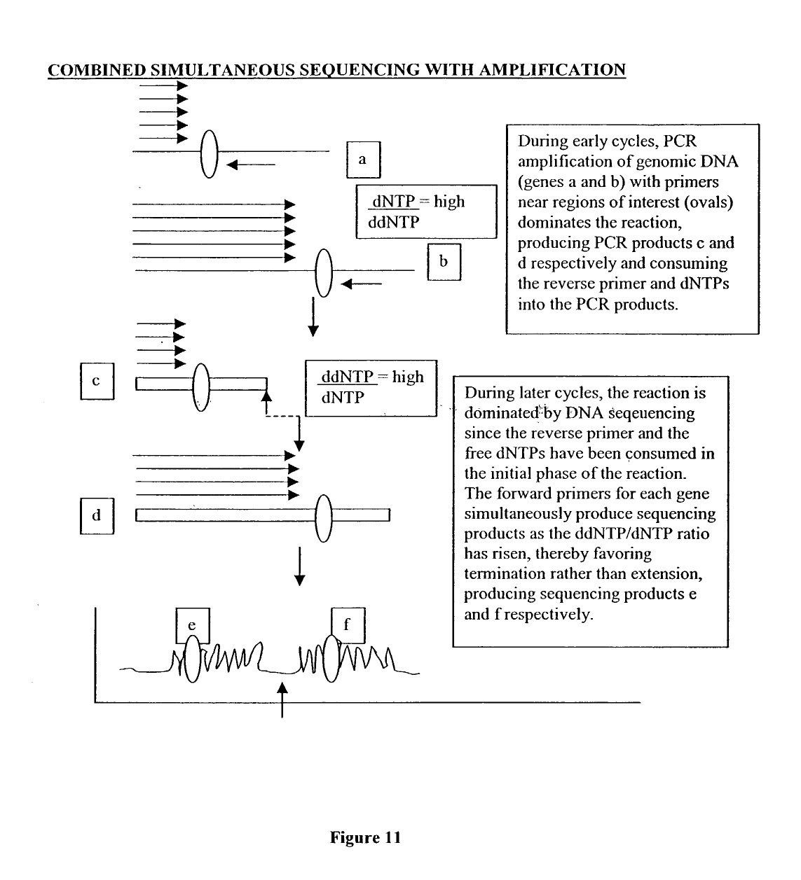

FIG. 11 shows a schematic of combined PCR and sequencing of two gene products simultaneously. In this Figure, two genes are shown, but this example is merely for illustrative purposes only and is not meant to construe or limit the invention in any way. The top third of the figure (early cycles) demonstrates the two targets (a and b), and their corresponding primers. In each case, the forward primer is present at five-fold increased molar ratio. Prior to the beginning of the reaction, the dNTP/ddNTP concentration is high because the reaction has been supplemented with additional dNTPs. In the middle panel, PCR has occurred (products c and d respectively) which results in a decrease in the concentration of the reverse primer and in the dNTP concentration. This raises the relative ratio of ddNTPs/dNTPs, thereby favoring termination (sequencing) in subsequent cycles. In the lower panel, these products may now be seen as products, e and f, respectively.

The oligonucleotide primers are selected to be “substantially” complementary to the different strands of each specific sequence to be amplified. This means that the primers must be sufficiently complementary to hybridize with their respective strands. The primer sequence therefore need not reflect the exact sequence of the template to which it binds. For example, a non-complementary nucleotide fragment may be attached to the 5′-end of the primer, with the remainder of the primer sequence being complementary to the template strand. Non-complementary sequences include the poly thymidine tails so that one of the primers is longer than the other primers to prevent superimposition during the analysis phase.

The primers may also be modified by conjugate molecules to further increase the binding affinity and hybridization rate of these oligonucleotides to a target. Such conjugate molecules may include, by way of example, cationic amines, intercalating dyes, antibiotics, proteins, peptide fragments, and metal ion complexes. Common cationic amines include, for example, spermine and spermidine, i.e. polyamines. Intercalating dyes known in the art include, for example, ethidium bromide, acridines and proflavine. Antibiotics which can bind to nucleic acids include, for example, actinomycin and netropsin. Proteins capable of binding to nucleic acids include, for example, restriction enzymes, transcription factors, and DNA and RNA modifying enzymes. Peptide fragments capable of binding to nucleic acids may contain, for example, a SPKK (serine-proline-lysine (arginine)-lysine (arginine)) motif, a KH motif or a RGG (arginine-glycine-glycine) box motif. See, e.g., Suzuki, EMBO J., 8:797-804 (1989); and Bund, et al., Science, 265:615-621 (1994). Metal ion complexes which bind nucleic acids include, for example, cobalt hexamine and 1,10-phenanthroline-copper. Oligonucleotides represent yet another kind of conjugate molecule when, for example, the resulting hybrid includes three or more nucleic acids. An example of such a hybrid would be a triplex comprised of a target nucleic acid, an oligonucleotide probe hybridized to the target, and an oligonucleotide conjugate molecule hybridized to the primers. Conjugate molecules may bind to the primers by a variety of means, including, but not limited to, intercalation, groove interaction, electrostatic binding, and hydrogen bonding. Those skilled in the art will appreciate other conjugate molecules that can be attached to the modified primers of the present invention. See, e.g., Goodchild, Bioconjugate Chemistry, 1(3):165-187 (1990). Moreover, a conjugate molecule can be bound or joined to a nucleotide or nucleotides either before or after synthesis of the oligonucleotide containing the nucleotide or nucleotides.

The invention thus provides methods for increasing the both the avidity of binding and the hybridization rate between a primer and its target nucleic acid by utilizing primer molecules having one or more modified nucleotides, preferably a cluster of about 4 or more, and more preferably about 8, modified nucleotides. In preferred embodiments, the modifications comprise 2′ modifications to the riboftiranosyl ring. In most preferred embodiments the modifications comprise a 2′-O-methyl substitution. Other examples of modifications can include nucleobases such as for example, the naturally occurring nucleobases adenine (A), guanine (G), cytosine (C), thymine (T) and uracil (U) as well as non-naturally occurring nucleobases such as xanthine, diaminopurine, 8-oxo-N ⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, Nucleic Acids Research, 1997, vol. 25, pp 4429-4443. The term “nucleobase” thus includes not only the known purine and pyrimidine heterocycles, but also heterocyclic analogues and tautomers thereof. It should be clear to the person skilled in the art that various nucleobases which previously have been considered “non-naturally occurring” have subsequently been found in nature. Any nucleobase may also have substitutions which do not hinder the combined amplification and sequencing reaction as described herein.

In accordance with the present invention, it is also an object to provide for increasing the rate of hybridization of a single-stranded oligonucleotide or primer to a target nucleic acid through the incorporation of a plurality of modified nucleotides into the oligonucleotide. An increased rate of hybridization accomplished in this manner would occur over and above the increase in hybridization kinetics accomplished by raising the temperature, salt concentration and/or the concentration of the nucleic acid reactants. For example, Helper oligonucleotides may be used. Helper oligonucleotides are generally unlabeled and can be used in conjunction with desired primers of the present invention to increase the primer's Tₘ and hybridization rate by “opening up” target nucleotide sequence regions which may be involved in secondary structure, thus making these regions available for hybridization with the primer.

In light of the present disclosure, those of skill in the art will easily recognize that using modified helper oligonucleotides which will hybridize with the target nucleic acid at an increased rate over their unmodified counterparts can lead to even greater hybridization rates of the primer to their target. Thus, methods and compositions for detecting oligonucleotides employing such modified helper oligonucleotides are intended to be encompassed within the scope of this invention.

As used herein, the term “Tₘ” refers to the mid-point melting temperature at which two nucleic acid polymers are found entirely bound and entirely separate. It should be appreciated that the actual value will vary in accord with the hybridization solution used. The Tₘ can either be calculated by computer based upon their sequences or empirically determined by experimental determination.

A functional measure of sequence identity that is used to assess similarity of sequences is the ability of a particular nucleotide molecule to hybridize with a second nucleotide under defined conditions. As used herein, “hybridization” includes any process by which a strand of a nucleic acid joins with a complementary strand through base-pairing. Thus, strictly speaking, the term refers to the ability of the primer to bind to the target nucleic acid sequence, or vice-versa.

Hybridization conditions are based on the melting temperature (Tₘ) of the nucleic acid binding complex or primer and are typically classified by degree of “stringency” of the conditions under which hybridization is measured. (Ausubel, et al., 1990). For example, “maximum stringency” typically occurs at about Tₘ-5% C. (5% below the Tₘ of the nucleic acid binding complex); “high stringency” at about 5-10% below the Tₘ; “intermediate stringency” at about 10-20% below the Tₘ of the nucleic acid binding complex; and “low stringency” at about 20-25% below the Tₘ. Functionally, maximum stringency conditions may be used to identify sequences having strict identity or near-strict identity with the primers; while high stringency conditions are used to identify sequences having about 80% or more sequence identity with the primers.

As used herein, the phrase “target nucleic acid” may refer to a nucleic acid polymer that is sought to be copied. The “target nucleic acid(s)” can be isolated or purified from a cell, bacterium, protozoa, fungus, plant, animal, etc. Alternatively, the “target nucleic acid(s)” can be contained in a lysate of a cell, bacterium, protozoa, fungus, plant, animal, etc.

If the sample to be used is of RNA, for example, use for diagnostic assays wherein the infectious agent is a retrovirus or any other organism that has an RNA genome. In such cases, preferred helper oligonucleotides have modifications which give them a greater avidity towards RNA than DNA. In a preferred embodiment, such modifications include a cluster of at least about 4 2′-O-methyl nucleotides. In a particularly preferred embodiment, such modifications would include a cluster of about 8 2′-O-methyl nucleotides.

Other cases whereby it is important to determine RNA expression levels, is in cancer. Use of the present invention allows for rapid diagnosis due to its simultaneous sequencing (SimulSeq) and/or AmpliSeq methodology. It is known that the processes of transformation and tumor progression are associated with changes in the levels of messenger RNA species (Slamon et al., 1984; Sager et al., 1993; Mok et al., 1994; Watson et al., 1994). Recently, a variation on polymerase chain reaction (PCR) analysis, known as RNA fingerprinting or differential display PCR, has been used to identify messages differentially expressed in ovarian or breast carcinomas (Liang et al., 1992; Sager et al., 1993; Mok et al., 1994; Watson et al., 1994). By using arbitrary primers to generate “fingerprints” from total cell RNA, followed by separation of the amplified fragments by high resolution gel electrophoresis, it is possible to identify RNA species that are either up-regulated or down-regulated in cancer cells. Results of these studies indicate the presence of several markers of potential utility for diagnosis of breast or ovarian cancer, including a6-integrin (Sager et al., 1993), DESTOO1 and DEST002 (Watson et al., 1994), and LF4.0 (Mok et al., 1994).

As used herein, “sample” or “test sample”, may refer to any source used to obtain nucleic acids for SimulSeq or AmpliSeq. A test sample is typically anything suspected of containing a target sequence. Test samples can be prepared using methodologies well known in the art such as by obtaining a specimen from an individual and, if necessary, disrupting any cells contained thereby to release target nucleic acids. These test samples include biological samples which can be tested by the methods of the present invention described herein and include human and animal body fluids such as whole blood, serum, plasma, cerebrospinal fluid, sputum, bronchial washing, bronchial aspirates, urine, lymph fluids and various external secretions of the respiratory, intestinal and genitourinary tracts, tears, saliva, milk, white blood cells, myelomas and the like; biological fluids such as cell culture supernatants; tissue specimens which may be fixed; and cell specimens which may be fixed. “Purified product” may refer to a preparation of the product which has been isolated from the cellular constituents with which the product is normally associated and from other types of cells which may be present in the sample of interest.

Any DNA sample may be used in practicing the present invention, including without limitation eukaryotic, prokaryotic and viral DNA. In a preferred embodiment, the target DNA represents a sample of genomic DNA isolated from a patient. This DNA may be obtained from any cell source or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at the site of infection or inflammation. DNA is extracted from the cell source or body fluid using any of the numerous methods that are standard in the art. It will be understood that the particular method used to extract DNA will depend on the nature of the source. The preferred amount of DNA to be extracted for use in the present invention is at least 5 pg (corresponding to about 1 cell equivalent of a genome size of 4×10⁹ base pairs).

As mentioned previously, any amplification procedure can be used, for example, multiplex PCR, LCR, RT-PCR, RCA and the like. “Amplification”, as used herein, refers to any in vitro process for increasing the number of copies of a nucleotide sequence or sequences, i.e., creating an amplification product which may include, by way of example additional target molecules, or target-like molecules or molecules complementary to the target molecule, which molecules are created by virtue of the presence of the target molecule in the sample. These amplification processes include but are not limited to multiplex PCR, Rolling Circle PCR, ligase chain reaction (LCR) and the like. In a situation where the target is a nucleic acid, an amplification product can be made enzymatically with DNA or RNA polymerases or transcriptases. Nucleic acid amplification results in the incorporation of nucleotides into DNA or RNA. As used herein, one amplification reaction may consist of many rounds of DNA replication. PCR is an example of a suitable method for DNA amplification. For example, one PCR reaction may consist of 30-100 “cycles” of denaturation and replication.

The earliest method for DNA amplification was the polymerase chain reaction (PCR) which operated only on linear segments of DNA and produced linear segments using specific primer sequences for the 5′- and 3′-ends of a segment of DNA whose amplification was desired. As an improvement on this method, linear rolling circle amplification (LRCA) uses a target DNA sequence that hybridizes to an open circle probe to form a complex that is then ligated to yield an amplification target circle and a primer sequence and DNA polymerase is added. The amplification target circle (ATC) forms a template on which new DNA is made, thereby extending the primer sequence as a continuous sequence of repeated sequences complementary to the ATC but generating only about several thousand copies per hour. An improvement on LRCA is use of exponential RCA (ERCA) with additional priming sequences that bind to the replicated ATC-complement sequences to provide new centers of amplification, thereby providing exponential kinetics and greatly increased amplification. Exponential rolling circle amplification (ERCA) employs a cascade of strand displacement reactions but is limited to use of the initial single stranded RCA product as a template for further DNA synthesis using individual single stranded primers that attach to said product but without additional rolling circle amplification.

Each of these methods makes use of one or more oligonucleotide primers or splice templates able to hybridize to or near a given nucleotide sequence of interest. After hybridization of the primer, the target-complementary nucleic acid strand is enzymatically synthesized, either by extension of the 3′ end of the primer or by transcription, using a promoter-primer or a splice template. In some amplification methods, such as PCR, rounds of primer extension by a nucleic acid polymerizing enzyme is alternated with thermal denaturation of complementary nucleic acid strands. Other methods, such as those of WO91/02818, Kacian and Fultz, U.S. Pat. No. 5,480,783; McDonough, et al., WO 94/03472; and Kacian, et al., WO 93/22461, are isothermal transcription-based amplification methods.

In each amplification method, however, side reactions caused by hybridization of the primer to non-target sequences can reduce the sensitivity of the target-specific reaction. These competing “mismatches” may be reduced by raising the temperature of the reaction. However, raising the temperature may also lower the amount of target-specific primer binding as well.

Thus, according to this aspect of the invention, primers having high target affinity, and comprising modified nucleotides in the target binding region, may be used in nucleic acid amplification methods to more sensitively detect and amplify small amounts of a target nucleic acid sequence, by virtue of the increased temperature, and thus the increased rate of hybridization to target molecules, while reducing the degree of competing side-reactions (cross-reactivity) due to non-specific primer binding. Preferred oligonucleotides contain at least one cluster of modified bases, but less than all nucleotides are modified in preferred oligonucleotides.

In another preferred embodiment, modified oligonucleotide primers are used in a nucleic acid amplification reaction in which a target nucleic acid is RNA. See, e.g., Kacian and Fultz, supra. The target may be the initially present nucleic acid in the sample, or may be an intermediate in the nucleic acid amplification reaction. In this embodiment, the use of preferred 2′-modified primers, such as oligonucleotides containing 2′-O-methyl nucleotides, permits their use at a higher hybridization temperature due to the relatively higher Tₘ conferred to the hybrid, as compared to the deoxyoligonucleotide of the same sequence. Also, due to the preference of such 2′-modified oligonucleotides for RNA over DNA, competition for primer molecules by non-target DNA sequences in a test sample may also be reduced. Further, in applications wherein specific RNA sequences are sought to be detected amid a population of DNA molecules having the same (assuming U and T to be equivalent) nucleic acid sequence, the use of modified oligonucleotide primers having kinetic and equilibrium preferences for RNA permits the specific amplification of RNA over DNA in a sample.

“Amplification products”, “amplified products” “PCR products” or “amplicons” comprise copies of the target sequence and are generated by hybridization and extension of an amplification primer. This term refers to both single stranded and double stranded amplification primer extension products which contain a copy of the original target sequence, including intermediates of the amplification reaction.

“Target” or “target sequence” may refer to nucleic acid sequences to be amplified. These include the original nucleic acid sequence to be amplified, its complementary second strand and either strand of a copy of the original sequence which is produced in the amplification reaction. The target sequence may also be referred to as the template for extension of hybridized amplification primers.

“Nucleotide” as used herein, is a term of art that refers to a base-sugar-phosphate combination. Nucleotides are the monomeric units of nucleic acid polymers, i.e. of DNA and RNA. The term includes ribonucleoside triphosphates, such as rATP, rCTP, rGTP, or rUTP, and deoxyribonucleotide triphosphates, such as dATP, dCTP, dUTP, dGTP, or dTTP. A “nucleoside” is a base-sugar combination, i.e. a nucleotide lacking phosphate. It is recognized in the art that there is a certain interchangeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e. dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

The term “nucleic acid” is defined to include DNA and RNA, and their analogs, and is preferably DNA. Further, the methods of the present invention are not limited to the detection of mRNAs. Other RNAs that may be of interest include tRNAs, rRNAs, and snRNAs.

“Incorporating” as used herein, means becoming part of a nucleic acid polymer.

“Terminating” as used herein, means causing a treatment to stop. The term includes means for both permanent and conditional stoppages. For example, if the treatment is enzymatic, a permanent stoppage would be heat denaturation; a conditional stoppage would be, for example, use of a temperature outside the enzyme's active range. Preferred methods of termination include the use of abasic regions. It is also expedient to use deoxyribonucleoside triphosphates as chain termination molecules which are modified at the 3′ position of the deoxyribose in such a way that they have no free OH group but are nevertheless accepted as a substrate by the polymerase. Examples of such chain termination molecules are 3′ fluoro, 3′-O-alkyl and 3′H-modified deoxyribonucleosides. 3′-H-modified deoxyribonucleotides are preferably used as chain termination molecules i.e. dideoxyribonucleoside triphosphates (ddNTP). It is preferable to use unlabeled chain termination molecules in the method according to the invention but it is also possible to use labeled chain termination molecules as known to a person skilled in the art. Any type of termination procedures are intended to fall within the scope of this term.

“Oligonucleotide” as used herein refers collectively and interchangeably to two terms of art, “oligonucleotide” and “polynucleotide”. Note that although oligonucleotide and polynucleotide are distinct terms of art, there is no exact dividing line between them and they are used interchangeably herein. An oligonucleotide is said to be either an adapter, adapter/linker or installation oligonucleotide (the terms are synonymous) if it is capable of installing a desired sequence onto a predetermined oligonucleotide. An oligonucleotide may serve as a primer unless it is “blocked”. An oligonucleotide is said to be “blocked,” if its 3′ terminus is incapable of serving as a primer.

The term “probe” refers to a strand of nucleic acids having a base sequence substantially complementary to a target base sequence. Typically, the probe is associated with a label to identify a target base sequence to which the probe binds, or the probe is associated with a support to bind to and capture a target base sequence. Two fundamental ways of generating oligonucleotide arrays include synthesizing the oligonucleotides on the solid phase in their respective positions; and synthesizing apart from the surface of the array matrix and attaching later are well known in the art and are incorporated herein by reference. (Southern et al., Genomics, 13:1008-1017(1992); Southern et al., WO89/10977). An array constructed with each of the oligonucleotides in a separate cell can be used as a multiple hybridization probe to examine the homologous sequence.

“Oligonucleotide-dependent amplification” as used herein refers to amplification using an oligonucleotide or polynucleotide or probe to amplify a nucleic acid sequence. An oligonucleotide-dependent amplification is any amplification that requires the presence of one or more oligonucleotides or polynucleotides or probes that are two or more mononucleotide subunits in length and that end up as part of the newly-formed, amplified nucleic acid molecule.

“Primer” as used herein refers to a single-stranded oligonucleotide or a single-stranded polynucleotide that is extended by covalent addition of nucleotide monomers during amplification. Nucleic acid amplification often is based on nucleic acid synthesis by a nucleic acid polymerase. Many-such polymerases require the presence of a primer that can be extended to initiate such nucleic acid synthesis. Here through the selection of primers, modified or otherwise, which determine the average molecular weight of the DNA segments (or size), the result can be achieved that the variations of size or molecular weights for the DNA segments formed by the various primer pairs only prevents superimposition or overlap. In multigene, unidirectional SimulSeq methods, long primers (or primers which when analyzed appear long) are employed to prevent superimposition of sequencing products. In bidirectional SimulSeq, bi-directional AmpliSeq or unidirectional AmpliSeq, the reverse primer suitably possesses two features: the primer is either long or modified to appear long and the primer possesses a modification inhibiting synthesis past a certain point (e.g. an abasic region). This permits the same molecule to possess both priming capability (from its complementary region), prevents full extension down the primer, and produces larger products of its own.

As used herein, “uni-directional” refers to the sequencing of a nucleic acid in a 5′ to 3′ direction of either strand of nucleic acid.

As used herein, “bi-directional” refers to the sequencing of a nucleic acid in a 5′ to 3′ direction of a double-stranded nucleic acid or complementary strand of a single stranded nucleic acid molecule.

“Primer dimer” is an extraneous DNA or an undesirable side product of PCR amplification which is thought to result from nonspecific interaction amplification primers. Primer dimers not only reduce the yield of the desired PCR product but they also compete with the genuine amplification products. Primer dimer as the name implies is a double stranded PCR product consisting of two primers and their complementary sequences. However, the designation is somewhat misleading because analysis of these products indicates that additional bases are inserted between the primers. As a result, a fraction of these artifacts may be due to spurious nonspecific amplification of similar but distinct primer binding regions that are positioned in the immediate vicinity.

“Stringency” is meant the combination of conditions to which nucleic acids are subject that cause the duplex to dissociate, such as temperature, ionic strength, and concentration of additives such as formamide. Conditions that are more likely to cause the duplex to dissociate are called “higher stringency”, e.g. higher temperature, lower ionic strength and higher concentration of formamide.

The phrase “hybridizing conditions” and its grammatical equivalents, when used with a maintenance time period, indicates subjecting the hybridization reaction admixture, in context of the concentration of the reactants and accompanying reagents in the admixture, to time, temperature, pH conditions sufficient to allow the polynucleotide probe to anneal with the target sequence, typically to form the nucleic acid duplex. Such time, temperature and pH conditions required to accomplish the hybridization depend, as is well known in the art on the length of the polynucleotide probe to be hybridized, the degree of complementarity between the polynucleotide probe and the target, the guanidine and cytosine content of the polynucleotide, the stringency of the hybridization desired, and the presence of salts or additional reagents in the hybridization reaction admixture as may affect the kinetics of hybridization. Methods for optimizing hybridization conditions for a given hybridization reaction admixture are well known in the art.

The term “label” refers to a molecular moiety capable of detection including, by way of example, without limitation, radioactive isotopes, enzymes, luminescent agents, dyes, and detectable intercalating agents. Any suitable means of detection may be employed, thus, the label maybe an enzyme label, a fluorescent label, a radioisotopic label, a chemiluminescent label, etc. Examples of suitable enzyme labels include alkaline phosphatase, acetylcholine esterase, α-glycerol phosphate dehydrogenase, alkaline phosphatase, asparaginase, β-galactosidase, catalase, δ-5-steroid isomerase, glucose oxidase, glucose-6-phosphate dehydrogenase, luciferase, malate dehydrogenase, peroxidase, ribonuclease, staphylococcal nuclease, triose phosphate isomerase, urease, and yeast alcohol dehydrogenase. Examples of suitable fluorescent labels include fluorescein label, an isothiocyanate label, a rhodamine label, a phycoerythrin label, a phycocyanin label, an allophycocyanin label, an o-phthaldehyde label, a fluorescamine label, 5,6-carboxymethyl fluorescein, Texas red, nitrobenz-2-oxa-1,3-diazol-4-yl (NBD), coumarin, dansyl chloride, and rhodamine. Preferred fluorescent labels are fluorescein (5-carboxyfluorescein-N-hydroxysuccinimide ester) and rhodamine (5,6-tetramethyl rhodamine), etc. Examples of suitable chemiluminescent labels include luminal label, an aromatic acridinium ester label, an imidazole label, an acridinium salt label, an oxalate label, a luciferin label an aequorin label. Alternatively, the sample may be labeled with non-radioactive label such as biotin. The biotin labeled probe is detected via avidin or streptavidin through a variety of signal generating systems known in the art. Labeled nucleotides are preferred form of detection label since they can be directly incorporated into the products of PCR during synthesis. Examples of detection labels that can be incorporated into amplified DNA include nucleotide analogs such as BrdUrd (Hoy and Schimke, Mutation Research, 290:217-230 (1993)), BrUTP (Wansick et al., J. Cell Biology, 122:283-293 (1993)) and nucleotides modified with biotin (Langer et al., Proc. Natl. Acad. Sci. USA, 78:6633 (1981)) or with suitable haptens such as digoxygenin (Kerkhof, Anal. Biochem., 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is Cyanine-5-dUTP or BrdUrd (BUDR triphosphate, Sigma), and a preferred nucleotide analog detection label is Biotin-16-uridine-5′-triphosphate (Biotin-16-dUTP, Boehringher Mannheim).

The term “agent” is used in a broad sense, in reference to labels, and includes any molecular moiety which participates in reactions which lead to a detectable response.

The term “support” refers to conventional supports such as beads, particles, dipsticks, fibers, filters, membranes and silane or silicate supports such as glass. In addition, support refers to porous or non-porous water insoluble material. The support can be hydrophilic or capable of being rendered hydrophilic and includes inorganic powders such as silica, magnesium sulfate and alumina; natural polymeric materials, particularly cellulosic materials and materials derived from cellulose, such as fiber containing papers, e.g., filter paper and chromatographic paper; synthetic or modified naturally occurring polymers such as nitrocellulose, cellulose acetate, poly(vinyl) chloride, polyacrylamide, crosslinked dextran, agarose, polyacrylate, polyethylene, polypropylene, poly(4-methylbutene), polystyrene, polymethacrylate, poly(ethylene terephthalate), nylon and polyvinyl butyrate. These materials can be used alone or in conjunction with other materials such as glass, ceramics, metals and the like.

Joining of the immobilized oligonucleotide to the solid support may be accomplished by any method that will continue to bind the immobilized oligonucleotide throughout the assay steps. Additionally, it is important that when the solid support is to be used in an assay, it be essentially incapable, under assay conditions, of the non-specific binding or adsorption of non-target oligonucleotides or nucleic acids.

Common immobilization methods include binding the nucleic acid or oligonucleotide to nitrocellulose, derivatized cellulose or nylon and similar materials. The latter two of these materials form covalent interactions with the immobilized oligonucleotide, while the former binds the oligonucleotides through hydrophobic interactions. When using these materials it is important to use a “blocking” solution, such as those containing a protein, such as bovine serum albumin (BSA), or “carrier” nucleic acid, such as salmon sperm DNA, to occupy remaining available binding sites on the solid support before use in the assay.

Other immobilization methods may include the use of a linker arm, for example, N-hydroxysuccinamide (NHS) and its derivatives, to join the oligonucleotide to the solid support. As mentioned, common solid supports in such methods are, without limitation, silica, polyacrylamide derivatives and metallic substances. In such a method, one end of the linker may contain a reactive group (such as an amide group) which forms a covalent bond with the solid support, while the other end of the linker contains another reactive group which can bond with the oligonucleotide to be immobilized. In a particularly preferred embodiment, the oligonucleotide will form a bond with the linker at its 3′ end. The linker is preferably substantially a straight-chain hydrocarbon which positions the immobilized oligonucleotide at some distance from the surface of the solid support. However, non-covalent linkages, such as chelation or antigen-antibody complexes, may be used to join the oligonucleotide to the solid support.

The phrase “electrophoretic separation” and similar terms typically can be any electrophoresis method known to those skilled in the art. Preferably, the electrophoretic separation is accomplished by high resolution slab gel electrophoresis. More preferably, the electrophoretic separation is accomplished by capillary electrophoresis.

Typically, the hybridization product to be amplified, functions in PCR as a primed template comprised of polynucleotide as a primer hybridized to a target nucleic acid as a template. In PCR, the primed template is extended to produce a strand of nucleic acid having a nucleotide sequence complementary to the template, i.e., template complement. Through a series of primer extension reactions, an amplified nucleic acid product is formed that contains the specific nucleic acid sequence complementary to the hybridization product.

If the template whose complement is to be produced is in the form of a double stranded nucleic acid, it is typically first denatured, usually by melting into single strands, such as single stranded DNA. The nucleic acid is then subjected to a first primer extension reaction by treating or contacting nucleic acid with a first polynucleotide synthesis primer having as a portion of its nucleotide sequence, a sequence selected to be substantially complementary to a portion of the sequence of the template. The primer is capable of initiating a primer extension reaction by hybridizing to a specific nucleotide sequence. Design of exemplary preferred primers is disclosed in the examples below.

Typically, for PCR applications, suitable primers are at least about 10 nucleotides in length, more typically at least about 15, 20, 25 or 30 nucleotides in length.

For use in unidirectional SimulSeq methods and systems of the invention preferred primers include those that contain a complementary region preferably at least or up to about 10, 15, 20, 25 or 30 bases in length and contain “tails” or non-complementary bases (or similar modification) which vary preferably from none to 50, 100, 200, 300, 400, 500, 600, 700, 800 or more bases. Such tails may be composes of any single nucleotide or nucleotide analog or mixture thereof.

For bi-directional SimulSeq and AmpliSeq methods of the invention, suitable primers include those that contain one typical (e.g. forward) PCR primer and one primer with modifications. The modified (e.g. reverse) primer includes a complementary region preferably having at least or up to about 10, 15, 20, 25 or 30 bases, a region that inhibits extension (e.g. an abasic region), and a tail of length preferably of 1 to 50, 100, 200, 300, 400, 500, 600, 700 or 800 or more bases which can be either complementary or non-complementary (e.g. thymidines) as may be desired for a specific application. Thymidine-containing tails are preferred for some applications. Unidirectional AmpliSeq may be accomplished using umnodified primers at a non-equal molar ratio which permit long unidirectional sequencing. Relative molar ratios are preferably about 5:1 or about 10:1 (other examples of molar ratios are about 20:1, 1:20, 1:10, or 1:5), though many molar ratios other than 1:1 are likely to work. The lower primer concentration is presumably sufficient to support PCR amplification during early cycles. Since it is present in limiting concentration, it is presumably either exhausted during PCR, or its sequencing products are relatively few in number such that only one primary sequence (that generated from the primer at high concentration) is seen in the electropherogram.

For combined SimulSeq and AmpliSeq, two gene targets are demonstrated, MTHFR and prothrombin. The choice of two gene targets and the specific genes example provided chosen is merely for illustrative purposes only and is not meant to construe or limit the invention in any way. In fact, successful sequencing of more than two targets simultaneously and combined with PCR is an obvious extension as will be appreciated by those in the art.

The primer extension reaction is accomplished by mixing an effective amount of the primer with the template nucleic acid, and an effective amount of nucleic acid synthesis inducing agent to form the primer extension reaction admixture. The admixture is maintained under polynucleotide synthesizing conditions for a time period, which is typically predetermined, sufficient for the formation of a primer extension reaction product.

The primer extension reaction is performed using any suitable method. Generally, it occurs in a buffered aqueous solution, preferably at a pH of about 7 to 9, most preferably, about 8. Preferably, a molar excess (for genomic nucleic acid, usually 10⁶:1 primer:template) of the primer is admixed to the buffer containing the template strand. A large molar excess is preferred to improve the efficiency of the process. For polynucleotide primers of about 10 to 30 nucleotides in length, a typical ratio is in the range of about 50 ng to 1 μg, preferably about 250 ng of primer per 100 ng to about 500 ng of mammalian genomic DNA or per 10 to 50 ng of plasmid DNA. As little as 50 ng of genomic DNA can be used.

The deoxyribonuclotide triphosphates (dNTPs), dATP, dCTP, dGTP and dUTP are also admixed to the primer extension reaction admixture to support the synthesis of primer extension products and depends on the size and number of products to be synthesized. Preferably, when uracil-N glycosylase enzyme is used according to the present invention, dUTP is used instead of dTTP so that subsequent treatment of the amplified product with UNG will result in the formation of oligonucleotide fragments. The invention includes the use of any analogue or derivative of dUTP which can be incorporated into the extension product and which is acted on by UNG to produce oligonucleotide fragments. The resulting solution is heated to about 95° C. for 5 min followed by 35 cycles of 95° C. for 45 secs, 55° C. for 45 secs, and 72° C. for 1 min followed by 72° C. for 10 min. After heating, the solution is allowed to cool to room temperature which is preferable for primer hybridization. To the cooled mixture is added an appropriate agent for inducing or catalyzing the primer extension reaction and the reaction is allowed to occur under conditions known in the art. The synthesis reaction may occur at from room temperature up to a temperature above which the inducing agent no longer functions efficiently. Thus, for example, if DNA polymerase is used as the inducing agent, the temperature is generally no greater than about 40° C. unless the polymerase is heat stable.

The inducing agent may be any compound or system which will function to accomplish the synthesis of the primer extension products, including enzymes. Suitable enzymes for this purpose include for example E. coli DNA polymerase I, Klenow fragment of E. coli DNA polymerase I, T4 DNA polymerase, T7 DNA polymerase, recombinant modified T7 DNA polymerase, other available DNA polymerase, reverse transcriptase and other enzymes including heat stable enzymes which will facilitate the combination of nucleotides in the proper manner to form the primer extension products which are complementary to each nucleic acid strand. Heat stable DNA polymerase is used in the most preferred embodiment by which PCR is conducted in a single solution in which the temperature is cycled. Representative heat stable polymerases are DNA polymerases isolated from Bacillus stearothermophilus (BioRad), Thermus Thermophilus (FINZYME, ATCC#27634), Thermus species (ATCC #31674), Thermus aquaticus strain TV 1151B (ATCC 25105), Sulfolobus acidocaldarius described by Bukrashuili et al. Biochem. Biophys. Acta 1008:102-7 (1989) and Elie et al. Biochem. Biophys. Acta 951:261-7 (1988) and Thermus filiformis (ATCC #43280). Particularly, the preferred polymerase is Taq DNA polymerase available from a variety of sources including Taq Gold (Applied Biosystems) Perkin Elmer Cetus (Norwalk, Conn.), Promega (Madison, Wis.) and Stratagene (La Jolla, Calif.) and AmpliTaq™ DNA polymerase, a recombinant Taq DNA polymerase available from Perkin-Elmer Cetus.

Generally, the synthesis will be initiated at the 3′ end of each primer and proceed in the 5′ direction along the template strand until the synthesis terminates, producing molecules of different lengths. There may be inducing agents, however, which initiate synthesis at the 5′ end and proceed in the above direction using the same process.

The primer extension reaction product is subjected to a second primer extension reaction by treating it with a second polynucleotide synthesis primer having a preselected nucleotide sequence. The second primer is capable of initiating the second reaction by hybridizing to a nucleotide sequence, preferably at least about 20 nucleotides in length and more preferably a predetermined amount thereof with the first product preferably, a predetermined amount thereof to form a second primer extension reaction admixture. The admixture is maintained under polynucleotide synthesizing conditions for a time period, sufficient for the formation of a second primer extension reaction product.

PCR is carried out simultaneously by cycling, i.e., performing in one admixture, the above described first and second primer extension reactions, each cycle comprising polynucleotide synthesis followed by denaturation of the double stranded polynucleotides formed. Methods and systems for amplifying a specific nucleic acid sequence are described in U.S. Pat. Nos. 4,683,195, 4,683,202 and 4,800,159, to Mullis et al; and the teachings in PCR Technology, Ehrlich, ed. Stockton press (1989); Faloona et al., Methods in Enzymol. 155:335-50 (1987): Polymerase Chain Reaction, Ehrlich, eds. Cold Spring Harbor Laboratories Press (1989), the contents of which are hereby incorporated by reference.

For purposes of this invention, “genetic diseases” are diseases which include specific deletions and/or mutations in genomic DNA from any organism, such as, e.g., sickle cell anemia, cystic fibrosis, α-thalassemia, β-thalassemia, muscular dystrophy, Tay-Sachs disease, cystic fibrosis (CF), and the like. Cancer includes, for example, RAS oncogenes. CF is one of the most common genetic diseases in Caucasian populations and more than 60 mutations have been found at this locus. Transforming mutations of RAS oncogenes are found quite frequently in cancers and more than 60 probes are needed to detect the majority of mutated variants. Analysis of CF and RAS mutants by conventional means is a difficult, complex and formidable task.

All of these genetic diseases may be detected by amplifying the appropriate sequence using SimulSeq or AmpliSeq.

Typically, UNG is added to the PCR products and incubated, preferably for about 30 min at about 37° C. for at least about 10 minutes. According to a preferred embodiment of this invention, hydrolysis of PCR products with about 1 unit of UNG for about 10 minutes at temperature of about 37° C. can render DNA incapable of being copied by DNA polymerase. UNG can be 95% heat killed at 95° C. for about 10 minutes. Typically, heat can be used to denature and cleave away unwanted uracil base, however, there are enzymes known to those skilled in the art that can also be used.

Uracil-DNA Glycosylase (UDG) or Uracil-N-Glycosylase (UNG) is an enzyme that catalyzes the release of free uracil from single stranded and double stranded DNA of greater than 6 base-pairs. This enzyme has found important use in the prevention of PCR template carry over contamination. PCR reactions are run in the presence of 2′-deoxyuridine 5′-triphosphate (dUTP) instead of 2′-deoxythymidine 5′-triphosphate (dTTP). The resulting dUTP-amplicon can be analyzed in a normal manner. However, to prevent the transfer of the amplicon into other PCR reactions, UNG is added to hydrolyze the amplicon into fragments. Such fragments are unable to participate in the next round of PCR, thus arresting unwanted contamination.

During the hydrolysis of the dUTP containing amplification product, an abundance of short oligonucleotide fragments are created. These oligonucleotides can be internally labeled (e.g., biotin-dCTP) during the course of the PCR reaction. The hybridization rate and signal intensity are enhanced using labeled oligo targets which are shorter than the full length PCR targets. The fragmentation pattern can also be predicted such that probes are designed for improved probe-target interaction.

The hybridization reaction mixture is maintained in the contemplated method under hybridizing conditions for a time period sufficient for the polynucleotide probe to hybridize to complementary nucleic acid sequences present in the sample to form a hybridization product, i.e., a complex containing probe and target nucleic acid.

Typical hybridizing conditions include the use of solutions buffered to pH values between 4 and 9, and are carried out at temperatures from 18° C. to 75° C., preferably at least about 22° C. to at least about 37° C., more preferably at least about 37° C. and for time periods from at least 0.5 seconds to at least 24 hours, preferably 30 min, although specific hybridization conditions will be dependent on the particular primer used.

Analysis of the SimulSeq and AmpliSeq reactions are suitably conducted in a single well in a gel or single capillary. The present invention is advantageous over the prior art which require that so called “simultaneously sequenced” products are divided prior to the reaction into different reaction vessels and analyzed in separate chambers in gels or capillaries. Preferred analysis methods include, but not limited to, a microcapillary electrophoresis device or array, for carrying out a size based electrophoresis of a sample. Microcapillary array electrophoresis generally involves the use of a thin capillary which may or may not be filled with a particular separation medium. Electrophoresis of a sample through the capillary provides a size based separation profile for the sample.

The use of microcapillary electrophoresis in size separation of nucleic acids has been reported in, e.g., Woolley and Mathies, Proc. Nat'l Acad Sci. USA (1994) 91:11348-11352, incorporated herein by reference in its entirety for all purposes. Microcapillary array electrophoresis generally provides a rapid method for size based sequencing, PCR product analysis and restriction fragment sizing. The high surface to volume ratio of these capillaries allows for the application of higher electric fields across the capillary without substantial heating, consequently allowing for more rapid separations. Furthermore, when combined with confocal imaging methods, these methods provide sensitivity in ranges which are comparable to the sensitivity of radioactive sequencing methods.

Microfabrication of capillary electrophoretic devices has been discussed in e.g., Jacobsen, et al., Anal. Chem. (1994) 66:1114-1118, Effenhauser, et al., Anal. Chem. (1994) 66:2949-2953, Harrison, et al. Science (1993) 261:895-897, Effenhauser, et al. Anal. Chem. (1993) 65:2637-2642, and Manz, et al., J. Chromatog. (1992) 593:253-258. Typically, these methods comprise photolithographic etching of micron scale capillaries in a silica or other crystalline substrate or chip.

In many capillary electrophoresis methods, silica capillaries are filled with an appropriate separation medium. Typically, a variety of separation media known in the art may be used in the microcapillary arrays. Examples of such media include, e.g., hydroxyethyl cellulose, polyacrylamide and the like. Generally, the specific gel matrix, running buffers and running conditions are selected to maximize the separation characteristics of the particular application, e.g., the size of the nucleic acid fragments, the required resolution, and the presence of native or denatured nucleic acid molecules.

The SimulSeq and AmpliSeq products can also be analyzed by out by separating the labeled nucleic acid fragments according to length. As discussed above, the present invention is advantageous in that the products are loaded into a single well without the requirement of separating the different reactions prior to analysis. This separation can be carried out according to all methods known in the state of the art e.g. by various electrophoretic (e.g. polyacrylamide gel electrophoresis) or chromatographic (e.g. HPLC) methods, a gel electrophoretic separation being preferred. Furthermore the labeled nucleic acids can be separated in any desired manner i.e. manually, semiautomatically or automatically, but the use of an automated sequencer is generally preferred. In this case the labeled nucleic acids can be separated in ultrathin plate gels of 20-500 μm preferably 100 μm thickness (see e.g. Stegemann et al., Methods in Mol. and Cell. Biol. 2 (1991), 182-184) or capillaries, as mentioned above. However, the sequence can also be determined in non-automated devices e.g. by a blotting method.

The invention is also useful for generating large volumes of nucleic acids for use in biochip arrays. In particular for detecting changes in gene expression, identification of the source of a cancerous gene or mutation, and the like.

Many biological functions are accomplished by altering the expression of various genes through transcriptional (e.g. through control of initiation, provision of RNA precursors, RNA processing, etc.) and/or translational control. For example, fundamental biological processes such as the cell cycle, cell differentiation and cell death, are often characterized by the variations in the expression levels of groups of genes.

Changes in gene expression also are associated with pathogenesis. For example, the lack of sufficient expression of functional tumor suppressor genes and/or the over expression of oncogene/proto-oncogenes could lead to tumorgenesis (Marshall, Cell, 64:313-326 (1991); Weinberg, Science, 254:1138-1146 (1991)). Thus, changes in the expression levels of particular genes (e.g. oncogenes or tumor suppressors) serve as signposts for the presence and progression of various diseases. For example, a bio chip allows for the attachment of several thousands of gene fragments, in assigned locations, to a glass slide or a silicon wafer to produce a “gene chip”. A single gene chip can contain up to 40,000 gene fragments for gene expression analysis. Gene fragments can be from any part of a gene or several parts of the same gene. In general, the gene fragments are composed of two different groups, experimental and control. The experimental group contains fragments of genes whose expression is going to be profiled. While the control group contains the fragments of genes for several positive and several negative control genes. Control genes provide the means to monitor the quality of an experiment and provide “landmarks” for the location of the genes attached to the glass or silicon support. Typically the gene fragments are arranged in a grid pattern, repeated several times to form a “super grid” so as to allow multiple data points for analysis and landmarks to locate specific gene fragments (Microarray Biochip Technology, ed. Mark Schena (Natick, M A: Eaton Publishing 2000).

The gene chip can be used to evaluate the differences in gene expression between untreated and treated cells. This is accomplished by differentially labeling the nucleic acids derived from the treated and untreated cells followed by sequence specific hybridization of the differentially labeled nucleic acids to the same gene chip. Conclusions and comparisons about the genes differentially expressed between the treated and untreated samples can be made after removal of the excess differentially labeled nucleic acid from the gene chip, data collection and data analysis (Microarray Biochip Technology, ed. Mark Schena (Natick, M A: Eaton Publishing 2000; Duggan, D. J., Bittner, M., Chen, Y., Meltzer, P. and Trent, J. M. (1999). Expression profiling using cDNA microarrays. Nature Genetics Vol. 21S, p. 10-14)).

Genes that are affected by the treatment of the cells are determined by comparing and identifying the differential gene expression between untreated and treated cells. For example, gene fragments having proportionally less labeled nucleic acid from the treated cells than from the untreated cells are said to have decreased expression or to have “repressed” gene expression. Whereas gene fragments that have proportionally more labeled nucleic acid from the treated cells than from the untreated cells are said to have increased expression or to have “induced” gene expression.

Analysis of a list containing the gene fragments, level of induction or repression or no change, and the function of the gene allows the identification of biological pathways that have altered gene expression patterns. Thus, the massive amount of genetic information provided by a single gene chip experiment allows the identification of biochemical pathways exhibiting altered gene expression patterns due to a specific drug treatment. A gene chip provides information about altered gene expression patterns from which the expression patterns of induction or repression of proteins can be deduced.

The term “biochip” as used herein, is a microarray chip comprised of gene fragments from any part of a gene or several parts of the same gene, whole genes, nucleic acids, proteins or fragments thereof, peptides or fragments thereof. The biochip can be comprised of any combinations of the above molecules in any pattern on the chip.

The term “pattern” as used herein, can be parallel horizontal or vertical lines, spots, circles, grids, checkered designs, or any other desired design.

Methods of forming high density arrays of oligonucleotides, peptides and other polymer sequences with a minimal number of synthetic steps are known. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668 which disclose methods of forming vast arrays of peptides, oligonucleotides and other molecules using for example, light-directed synthesis techniques. See also, Fodor et al., Science, 251:767-777 (1991). These procedures for synthesis of polymer arrays are now referred to as VLSIPS™ procedures. Using the VLSIPS™ approach, one heterogeneous array of polymers is converted through simultaneous coupling at a number of reaction sites, into a different heterogeneous array.

The development of VLSIPS™ technology is considered pioneering technology in the fields of combinatorial synthesis and screening of combinatorial libraries. In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′-photoprotected nucleoside phosphoramidite. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In the event that an oligonucleotide analogue with a polyamide backbone is used in the VLSIPS™ procedure, it is generally inappropriate to use phosphoramidite chemistry to perform synthetic steps, since the monomers do not attach to one another via a phosphate linkage. Instead, peptide synthetic methods are substituted. See, e.g. Pirrung et al., U.S. patent No. 5,143,854.

Peptide substituted nucleic acids are commercially available from e.g. Biosearch, Inc. (Bedford, Mass.) which comprise a polyamide backbone and the bases found in naturally occurring nucleosides. Peptide nucleic acids are capable of binding to nucleic acids with high specificity, and are considered “oligonucleotide analogues” for purposes of this disclosure.

In accord with the present invention, large arrays can be generated using presynthesized oligonucleotides generated by SimulSeq and/or AmpliSeq. The oligonucleotides are laid down in linear rows to form an array, which then can be divided or cut into strips, to form a number of smaller, uniform arrays. Strips from different arrays can be combined to form more complex composite arrays. In this way, both the efficiency of oligonucleotide attachment (or synthesis) is improved, and there is a significant increase in reproducibility of the arrays.

It is also a desired embodiment of the present invention to provide regions having varying widths and lengths of attached oligonucleotides. Each oligonucleotide can form an oligonucleotide strip that is longer than it is wide; that is, when hybridization to a target sequence occurs, a strip of hybridization occurs. This significantly increases the ability to distinguishing over non-specific hybridization and background effects when detection is via visualization, such as through the use of radioisotope detection. When other types of detection such as fluorescence is used, the length of the strip allows repeated detection reactions to be made, with or without slight variations in the position along the length of the strip. Averaging of the data points allows the minimization of false positives or position dependent noise such as dust, microdebris, etc.

Thus, the present invention also provides for oligonucleotide arrays comprising a solid support with a plurality of different oligonucleotide pools. By “plurality” herein is meant at least two different oligonucleotide species, with from about 10 to 1000 being preferred, and from about 50 to 500 being particularly preferred and from about 100-200 being especially preferred, although smaller or larger number of different oligonucleotide species may be used as well. As will be appreciated by those in the art, the number of oligonucleotides per array will depend in part on the size and composition of the array, as well as the end use of the array. Thus, for certain diagnostic arrays, only a few different oligonucleotide probes may be required; other uses such as cDNA analysis may require more oligonucleotide probes to collect the desired information.

The composition of the solid support may be anything to which oligonucleotides may be attached, preferably covalently, and will also depend on the method of attachment. Preferably, the solid support is substantially nonporous; that is, the oligonucleotides are attached predominantly at the surface of the solid support.

Accordingly, suitable solid supports include, but are not limited to, those made of plastics, resins, polysaccharides, silica or silica-based materials, functionalized glass, modified silicon, carbon, metals, inorganic glasses, membranes, nylon, natural fibers such as silk, wool and cotton, and polymers. In some embodiments, the material comprising the solid support has reactive groups such as carboxy, amino, hydroxy, etc., which are used for attachment of the oligonucleotides. Alternatively, the oligonucleotides are attached without the use of such functional groups, as is more fully described below. Polymers are preferred, and suitable polymers include, but are not limited to, polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate and polymethylpentene. Other preferred polymers include those well known in the art, see for example, U.S. Pat. No. 5,427,779.

The solid support has covalently attached oligonucleotides produced by SimulSeq or AmpliSeq. By “oligonucleotide” or “nucleic acid” or grammatical equivalents herein is meant at least two nucleotides covalently linked together. A nucleic acid of the present invention will generally contain phosphodiester bonds, although in some cases, a nucleic acid may have an analogous backbone, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate, phosphorodithioate, phosphoramidate, 0-methylphophoroamidite linkages (see Eckstein, Oligonucleotides and Analogues: A Practical Approach, Oxford University Press), peptide nucleic acid linkages (see Egholm, J. Am. Chem. Soc. 114:1895 (1992); Meier et al., Chem. Int. Ed. Engl. 31:1008 (1992); Nielsen, Nature, 365:566 (1993)) or morpholino-type backbones. These modifications of the ribose-phosphate backbone may be done to increase the stability and half-life of such molecules in physiological environments, or to increase the stability of the hybridization complexes (duplexes). Generally, the attached oligonucleotides are single stranded. The oligonucleotide may be DNA, both genomic and CDNA, RNA or a hybrid, where the oligonucleotide contains any combination of deoxyribo- and ribo-nucleotides, and any combination of uracil, adenine, thymine, cytosine and guanine, as well as other bases such as inosine, xanthine and hypoxanthine.

The length of the oligonucleotide, i.e. the number of nucleotides, can vary widely, as will be appreciated by those in the art. Generally, oligonucleotides of at least 6 to 8 bases are preferred, with oligonucleotides ranging from about 10 to 500 being preferred, with from about 20 to 200 being particularly preferred, and about 20 to 40 being especially preferred. Longer oligonucleotides are preferred, since higher stringency hybridization and wash conditions can be used, which decreases or eliminates non-specific hybridization. However, shorter oligonucleotides can be used if the array uses levels of redundancy to control the background, or utilizes more stable duplexes.

The arrays of the invention comprise at least two different covalently attached oligonucleotide species, with more than two being preferred. By “different” oligonucleotide herein is meant an oligonucleotide that has a nucleotide sequence that differs in at least one position from the sequence of a second oligonucleotide; that is, at least a single base is different. If the desired pattern is comprised of parallel lines, arrays can be made wherein not every strip contains an oligonucleotide. That is, when the solid support comprises a number of different support surfaces, such as fibers, for example, not every fiber must contain an oligonucleotide. For example, “spacer” fibers (or rows, when a single support surface is used) may be used to help alignment or detection. In a preferred embodiment, every row or fiber has a covalently attached oligonucleotide. In this embodiment, some rows or fibers may contain the same oligonucleotide, or all the oligonucleotides may be different. Thus, for example, it may be desirable in some applications to have rows or fibers containing either positive or negative controls, evenly spaced throughout the array, i.e., every nth fiber or row is a control. Similarly, any level of redundancy can be built into the array; that is, different fibers or rows containing identical oligonucleotides can be used.

The space between the oligonucleotide strips, or spots, etc, can vary widely, although generally is kept to a minimum in the interests of miniaturization. The space will depend on the methods used to generate the array; for example, for woven arrays utilizing fibers, the methodology utilized for weaving can determine the space between the fibers.

Each oligonucleotide pool or species is arranged in a desired pattern design, such as for example, a linear row to form an immobilized, distinct, oligonucleotide strip. By “distinct” herein is meant that each row is separated by some physical distance. By “immobilized” herein is meant that the oligonucleotide is attached to the support surface, preferably covalently. By “strip” herein is meant a conformation of the oligonucleotide species that is longer than it is wide. When the array comprises a number of different support surfaces, such as outlined above for fibers, each strip is a different fiber. However, the arrays can be arranged in any desired pattern.

In one embodiment, the solid support comprises a single support surface. That is, a plurality of different oligonucleotide pools are attached to a single support surface, in distinct linear rows, forming oligonucleotide strips. In a preferred embodiment, the linear rows or stripes are parallel to each other. However, any conformation of strips or desired patterns can be used as well. In one embodiment, there are preferably at least about 1 strip per millimeter, with at least about 2 strips per millimeter being preferred, and at least about 3 strips per millimeter being particularly preferred, although arrays utilizing from 3 to 10 strips, or higher, per millimeter also can be generated, depending on the methods used to lay down the oligonucleotides.

In an alternative embodiment, the solid support comprises a plurality of separate support surfaces that are combined to form a single array. In this embodiment, each support surface can be considered a fiber. Thus, the array comprises a number of fibers, each of which can contain a different oligonucleotide. That is, only one oligonucleotide species is attached to each fiber, and the fibers are then combined to form the array.

By “fiber” herein is meant an elongate strand. Preferably the fiber is flexible; that is, it can be manipulated without breaking. The fiber can have any shape or cross-section. The fibers can comprise, for example, long slender strips of a solid support that have been cut off from a sheet of solid support. Alternatively, and preferably, the fibers have a substantially circular cross section, and are typically thread-like. Fibers are generally made of the same materials outlined above for solid supports, and each solid support can comprise fibers with the same or different compositions.

The fibers of the arrays can be held together in a number of ways. For example, the fibers can be held together via attachment to a backing or support. This is particularly preferred when the fibers are not physically interconnected. For example, adhesives can be used to hold the fibers to a backing or support, such as a thin sheet of plastic or polymeric material. In a preferred embodiment, the adhesive and backing are optically transparent, such that hybridization detection can be done through the backing. In a preferred embodiment, the backing comprises the same material as the fiber; alternatively, any thin films or sheets can be used. Suitable adhesives are known in the art, and will resist high temperatures and aqueous conditions. Alternatively, the fibers can be attached to a backing or support using clips or holders. In an additional embodiment, for example when the fibers and backing comprise plastics or polymers that melt, the fibers are attached to the backing via heat treatment at the ends. The fibers, i.e., the separate support surfaces, plus the means to hold them together, together form the solid support.

In a preferred embodiment, the fibers are woven together to form woven fiber arrays. Thus, the array further comprises at least a third and a fourth fiber which are interwoven with the first and second fibers. In this embodiment, either or both of the weft (also sometimes referred to as the woof) and warp fibers contains covalently attached oligonucleotides.

If desired, the strips of different arrays can be placed adjacently together to form composite or combination arrays. A “composite” or “combination array” or grammatical equivalents is an array containing at least two strips from different arrays for a fiber array; the same types of composite arrays can be made from single support surface arrays. That is, one strip is from a first fiber array, and another is from a second fiber array. The second fiber array has at least one covalently attached oligonucleotide that is not present in said first array, i.e. the arrays are different.

The composite arrays can be made solely of alignment arrays, solely of woven arrays, or a combination of different types. The width and number of strips in a composite array can vary, depending on the size of the fibers, the number of fibers, the number of target sequences for which testing is occurring, etc. Generally, composite arrays comprise at least two strips. The composite arrays can comprise any number of strips, and can range from about 2 to 1000, with from about 5-100 being particularly preferred.

The strips of arrays in a composite array are generally adjacent to one another, such that the composite array is of a minimal size. However, there can be small spaces between the strips for facilitating or optimizing detection. Additionally, as for the fibers within an array, the strips of a composite array may be attached or stuck to a backing or support to facilitate handling.

Methods of making the oligonucleotide arrays of the present invention suitably may vary. In a preferred embodiment, oligonucleotides are synthesized using SimulSeq or AmpliSeq and then attached to the support surface, see for example, U.S. Pat. Nos. 5,427,779; 4,973,493; 4,979,959; 5,002,582; 5,217,492; 5,258,041 and 5,263,992. Briefly, coupling can proceed in one of two ways: a) the oligonucleotide is derivatized with a photoreactive group, followed by attachment to the surface; or b) the surface is first treated with a photoreactive group, followed by application of the oligonucleotide. The activating agent can be N-oxy-succinimide, which is put on the surface first, followed by attachment of a N-terminal amino-modified oligonucleotide, as is generally described in Amos et al., Surface Modification of Polymers by Photochemical Immobilization, The 17th Annual Meeting of the Society of Biomaterials, May 1991, Scottsdale Ariz. Thus, for example, a suitable protocol involves the use of binding buffer containing 50 mM sodium phosphate pH 8.3, 15% Na₂SO₄ and 1 mm EDTA, with the addition of 0.1-10 pM/μl of amino-terminally modified oligonucleotide. The sample is incubated for some time, from 1 second to about 45 minutes at 37° C., followed by washing (generally using 0.4 N NaOH/0.25% Tween-20), followed by blocking of remaining active sites with 1 mg/ml of BSA in PBS, followed by washing in PBS. The methods allow the use of a large excess of an oligonucleotide, preferably under saturating conditions; thus, the uniformity along the strip is very high.

The oligonucleotides can also be covalently attached to the support surface. In an additional embodiment, the attachment may be very strong, yet non-covalent. For example, biotinylated oligonucleotides can be made, which bind to surfaces covalently coated with streptavidin, resulting in attachment.

Oligonucleotides can be added to the surface in a variety of ways. In one method, the entire surface is activated, followed by application of the oligonucleotide pools in linear rows or any other desired pattern, with the appropriate blocking of the excess sites on the surface using known blocking agents such as bovine serum albumin. Alternatively, the activation agent can be applied in linear rows, followed by oligonucleotide attachment.

Application of the oligonucleotides can be done in several ways. In a preferred embodiment, the oligonucleotides are applied using ink jet technology, for example using a piezoelectric pump. In another method, the oligonucleotides are drawn, using for example a pen with a fine tip filled with the oligonucleotide solution. If a series or pattern of dots is desired, for example, a plotter pen may be used. In addition, patterns can be etched or scored into the surface to form uniform microtroughs, followed by filling of the microtrough with solution, for example using known microfluidic technologies.

Oligonucleotide arrays have a variety of uses, including the detection of target sequences, sequencing by hybridization, and other known applications (see for example Chetverin et al., Biotechnology, Vol. 12, November 1994, pp1034-1099, (1994)).

In a preferred embodiment, the arrays are used to detect target sequences in genes derived from a malignancy. The term “target sequence” or grammatical equivalents herein can mean a nucleic acid sequence on a single strand of nucleic acid. In some embodiments, a double stranded sequence can be a target sequence, when triplex formation with the probe sequence is done. The target sequence may be a portion of a gene, a regulatory sequence, genomic DNA, cDNA, mRNA, or others. It may be any length, with the understanding that longer sequences are more specific. As is outlined herein, oligonucleotides are made to hybridize to target sequences to determine the presence, absence, or relative amounts of the target sequence in a sample.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including but not limited to the β-actin gene, the transferrin receptor gene, the GAPDH gene and the like.

Similarly, arrays can be generated containing oligonucleotides designed to hybridize to mRNA sequences and used in differential display screening of different tissues, or for DNA indexing. In addition, the arrays of the invention can be formulated into kits containing the arrays and any number of reagents, such as PCR amplification reagents, labeling reagents, etc.

The following non-limiting examples are illustrative of the invention.

General Comments to Examples

The following materials and methods were employed in the examples below.

Materials and Methods

Polymerase Chain Reaction (PCR)

PCR was carried out in a 50 μl reactions containing a final concentration of 1×PCR Buffer (Applied Biosystems, Foster City, Calif.), 50 μM each dNTP, 1.25 U Taq Gold (Applied Biosystems), 0.01% gelatin and 0.2 μM each forward and reverse primer. The reaction mixture was subjected to 95° C. for 5 min followed by 35 cycles of 95° C. for 45 seconds, 55° C. for 45 seconds, and 72° C. for 1 min followed by 72° C. for 10 min. The PCR products were identified on 10% PAGE and then purified using QIAquick PCR Purification kit (Qiagen, Valencia, Calif.) according to the manufacturer's instructions. All oligonucleotides were synthesized and purified by Oligo's Etc. (Wilsonville, Oreg.). Following PCR, to some samples, 3 μl (1 U/μl) of uracil-N-glycosylase (UNG) (Life Technologies, Carlsbad, Calif.) was added to the 50 μl prothrombin PCR product, and incubated at 37° C. for 30 min. The enzyme was then heat inactivated by incubation at 94° C. for 10 min.

Cycle Sequencing

Cycle sequencing was performed using the BigDye™ version 2.0 or 3.0 Terminator Cycle Sequencing kit according to manufacturer's instructions (Applied Biosystems). Products were analyzed using an ABI Prism 3700 (Applied Biosystems).

Bi-directional Simultaneous Sequencing

Factor V forward primer, 5′-TGCCCACTGCTTAACAAGACCA-3′ (SEQ ID NO:11), and reverse primer, 5′-AAGGTTACTTCAAGGACAAAATAC-3′ (SEQ ID NO:12), were designed to amplify a 145 bp-product encompassing the mutation site. Forward sequencing primer was 5′-AGGACTACTTCTAATCTGGTAAG-3′ (SEQ ID NO:13). The reverse sequencing primer was identical to the reverse PCR primer with the 5′ addition of 4 abasic sites followed by 90 thymidines and was gel purified. Equal amounts of two sequencing primers were used.

Factor V Leiden RFLP

Factor V forward primer, 5′-TGCCCAGTGCTTAACAAGACCA-3′ (SEQ ID NO:1), and reverse primer, 5′-TGTTATCACACTGGTGCTAA-3′ (SEQ ID NO:2), were used as described by Bertina et al. (9). Amplified products were digested by MnlI, separated by PAGE and stained with ethidium bromide.

Amplification/Sequencing

Primers for bi-directional combined amplification/sequencing were identical to the sequencing primers described for bi-directional simultaneous sequencing. For unidirectional combined amplification/sequencing, the forward primer was identical to that used in the Factor V Leiden RFLP assay, and the reverse primer that was used in bi-directional combined amplification/sequencing with the tail extended to a total of 126 thymidines (total length 150 bases). Reactions were performed with 50-500 ng of genomic DNA, 0, 12.5, or 125 μM supplemental dNTPs in 20 μl reactions of BigDye™ version 2.0 Terminator Cycle Sequencing kit, and cycling conditions according to the manufacturer's instructions. Following combined PCR amplification/sequencing, the products were purified with spin columns (Biomax, Odenton, Md.) and analyzed on an ABI 3700.

### EXAMPLES

### Examples 1-4

To demonstrate simultaneous sequencing of multiple DNA targets, three genes were sequenced simultaneously. Factor V Leiden (Arg506Gln) (10), prothrombin (G20210 A) (11), and the methylenetetrahydrofolate reductase (MTHFR, Ala223Val) (12) mutations each result in an increased risk of thrombosis, and mutations in combination appear to have a synergistic effect on thrombosis risk (13). The experimental strategy is depicted in FIG. 1A. PCR of each gene was designed with the known mutation site near the distal end of the PCR strand to be sequenced. This design is important in simultaneous sequencing reactions so that sequencing products terminate shortly after the site of interest, allowing sequencing products generated from other targets to be detected downstream. PCR amplification of each gene was performed in separate PCR reactions using the primers listed in Table 1. The three PCR products were mixed at equal concentrations and simultaneously sequenced using a mixture of three forward sequencing primers (Table 1), one for each gene, in a single tube. The results of simultaneous sequencing of the three genes are shown in FIGS. 1B-E. During simultaneous sequencing, the 22 base MTHFR sequencing primer extends up to 42 bases to the end of the PCR product such that the largest MTHFR sequence product was 64 bases in length. A 69 base prothrombin sequencing primer was designed with 24 complementary bases tailed with an additional 45 thymidines on the 5′ end of the primer. This design creates a 6 base gap in sequencing products between the final MTHFR sequencing product (64 bases) and the beginning of prothrombin sequencing products (70 bases) making it easy to distinguish the two. Prothrombin sequence extends up to 39 bases to the end of the PCR product such that the final prothrombin sequence product is 108 bases. A 113 base Factor V sequencing primer was designed with 23 complementary bases tailed with an additional 90 thymidines on the 5′ end. This creates a gap between the final prothrombin sequencing product and Factor V sequencing products, which begin at 114 bases and continue up to 183 bases to the end of the PCR product. FIGS. 1B-D demonstrate simultaneous sequencing of the three prothrombotic genes on each of three patients heterozygous for Factor V Leiden (FIG. 1B), prothrombin (FIG. 1C), or MTHFR (FIG. 1D) mutations.

FIG. 1 shows the data obtained using SimulSeq for sequencing of three genes. (A) Experimental Design. PCR products (bars) for 3 different genes were designed such that the mutation site (indicated by a “*”) was near the distal end of the PCR strand to be sequenced. Sequencing primers (arrows) increasing in size with complimentary (solid) and non-complimentary (striped) bases were designed for each gene. The large sequencing primers were designed to be several bases longer than the largest sequencing product of the previous reaction with the shorter sequencing primer. This creates a “dead space” between the sequencing products of different reactions. The left ends of the PCR products are not shown (indicated with curved lines). Simultaneous sequencing of PCR products from the MTHFR, prothrombin (PROT), and factor V (FV) genes demonstrating (B) factor V Leiden, (C) prothrombin, and (D) MTHFR heterozygotes. The first bases detected are the result of MTHFR sequencing, which is followed by a dead space, the prothrombin sequencing products, a second dead space, and the factor V sequence products. Only the first—35 bases of factor V sequencing (which contains the Leiden mutation site) are shown. Shaded bars indicate the known mutation/polymorphic site for each gene; arrows demonstrate heterozygous sequence. (E) Use of UNG to eliminate sequence products resulting from the reverse PCR primer. Base sizes indicated are not accurate due to cropping of the figure.

An additional modification to this method is useful for obtaining even shorter stretches of sequence in order to permit larger numbers of sequencing reactions to be run simultaneously. This technique eliminates the majority of sequencing downstream of the mutation site since it reflects the known sequence of the reverse primer, providing no additional information. A prothrombin reverse PCR primer was designed that was identical to that used in FIGS. 1B-D except that two thymidines near the 3′ end of the primer were replaced with uracils (which should not limit its priming ability). After PCR, the prothrombin PCR products were treated with UNG and then mixed with MTHFR and Factor V PCR products, and simultaneously sequenced with the three sequencing primers as above. UNG treatment creates abasic sites in the prothrombin PCR products, which selectively terminate the prothrombin sequence at the beginning of the reverse primer (FIG. 1E). This technique could be employed to simultaneously acquire very short (e.g. 10-20 bases) segments of sequence from many different gene sequences, making simultaneous sequencing a viable method to detect a large panel of mutations or single nucleotide polymorphisms (SNPs).

### Examples 5-6

To obtain both forward and reverse sequence from a single gene product using simultaneous sequencing, the Factor V PCR reaction was re-designed such that the mutation site was located near one end of the 145 bp PCR product. A forward sequencing primer, 22 bases in length, was designed to yield up to 54 bases of sequencing (to the end of the PCR product). Also designed was a large reverse primer with 24 complementary bases, 56 non-coding thymidines and four abasic sites between the coding and non-coding bases. The abasic sites are important because products from the reverse primer can serve as templates for the forward primer. Without the reverse primer abasic sites, some forward primer sequencing products could terminate within the non-coding thymidine region of the reverse primer and be superimposed on those generated from the reverse primer. The experimental design is depicted in FIG. 2A. Bi-directional sequencing for both a Factor V wild-type homozygote and Leiden heterozygote is demonstrated in FIG. 2B. As shown, when the forward and reverse primers are used to cycle-sequence simultaneously, there is a short (˜5 base) gap between the end of the forward sequencing products and the beginning of the reverse sequence, making it easy to distinguish the two. The results of simultaneous forward and reverse sequencing correlate with the results of the standard RFLP assay (FIG. 2C).

The results of the above are shown in FIG. 2, which illustrates the use of bidirectional SimulSeq. (A) Experimental design of simultaneous forward and reverse sequencing. The rectangle represents the double stranded PCR product. The mutation site is indicated by a “*”. The forward and reverse sequencing primers are represented by arrows with the complimentary bases depicted as solid lines adjacent to the PCR product. In the reverse sequencing primer, the dots represent the abasic sites and the solid tail region of the primer, non-templated thymidines. (B) Results of simultaneous forward and reverse sequencing of homozygous wild type (WT/WT) and heterozygous Leiden mutant (WT/L) individuals. Shaded bars indicate the mutation site in both the forward and reverse sequence products. Arrows demonstrate heterozygous sequence. (C) Conventional RFLP assay for factor V Leiden mutation. Non-denaturing 10% polyacrylamide gel electrophoresis (PAGE) of PCR products following restriction digest with Mnl I and ethidium bromide staining. Homozygous wild type (WT/WT) amplicons have 2 digestion sites within the PCR product producing anticipated bands of 37 bp, 67 bp, and 163 bp. The Leiden mutation destroys one digestion site such that the 37 and 163 by bands are combined to produce an additional 200 by band in the heterozygous mutant (WT/L) sample. Molecular weight markers as designated.

### Examples 7-8

Standard cycle sequencing reactions containing genomic DNA and the Factor V forward and reverse primers, as described above, were performed. The anticipated PCR product is diagrammed in FIG. 3A. To support PCR, the reactions were supplemented with additional dNTPs at varying concentrations. Using this approach, early cycles should be dominated by PCR amplification (since the free deoxynucleotide concentration is relatively high), and later cycles by cycle sequencing (because depletion of free deoxynucleotides during PCR increases the relative di-deoxynucleotide concentration). Without deoxynucleotide supplementation, no discernable sequencing products were identified. With the addition of 12.5 μM or 125 μM deoxynucleotides, both forward and reverse sequencing products were generated (FIG. 3B). This strategy supports combined PCR and sequencing in single reactions.

Input genomic DNA concentrations ranging from 50 to 500 ng yielded approximately equivalent amounts of sequencing products. Combined amplification/sequencing technology has also been used to generate forward and reverse sequence data of the APC I1307K mutation. In order to generate long unidirectional sequencing (FIG. 3C), the Factor V combined amplification/sequencing reaction was re-designed by moving the forward primer further upstream of the Leiden mutation and lengthening the reverse primer tail to 126 thymidines. Therefore, combined amplification/sequencing reactions yield either bi-directional or long unidirectional sequence in combination with PCR amplification. The present invention likewise provides a method whereby one of skill in the art could design combined amplification/sequencing reactions to simultaneously amplify and sequence multiple genes at the same time.

The results obtained using AmpliSeq, as described above are shown in FIG. 3. (A) Anticipated PCR product generated during AmpliSeq. Forward and reverse primer sequences are shown as dark shading, and the rest of the PCR product as light shading. In the reverse primer, dots denote the abasic region and stripes, the non-templated thymidines. The mutation site is indicated by a “*”. (B) Bidirectional AmpliSeq results of a factor V wildtype homozygote. (C) Unidirectional AmpliSeq of a factor V wildtype homozygote. Shaded bars indicate the potential mutation site in sequencing products.

TABLE 1

Oligonucleotide primers used for PCR and sequencing.

| Gene | PCR Primers | Sequencing Primer |
|---|---|---|
| Factor V | (F)^{a} 5′-TGCCCAGTGCTTAACAAGACCA-3′ | 5′-T₉₀GGCTAATAGGACTACTTCTAATC-3′ |
| | (SEQ ID NO:1) | (SEQ ID NO:3)(gel purified) |
| | (R) 5′-TGTTATCACACTGGTGGTAA-3′ | |
| | (SEQ ID NO:2) | |
| Prothrombin | (F) 5′-GGATGGGAAATATGGCTTCTACAC-3′ | 5′-T₄₅-TAAAAACTATGGTTCCCAATAAAAG-3′ |
| | (SEQ ID NO:4) | (SEQ ID NO:6)(gel purified) |
| | (R) 5′-ATGAATAGCACTGGGAGCATTG-3′^{b} | |
| | (SEQ ID NO:5) | |
| MTHFR | (F) 5′-CTCCTGACTGTCATCCCTATTG-3′ | 5′-CCTGAAGCACTTGAAGGAGAAG-3′ |
| | (SEQ ID NO:7) | (SEQ ID NO:9) |
| | (R) 5′-AAAGAAAAGCTGCGTGATGATG-3′ | |
| | (SEQ ID NO:8) | |

^{a}(F) forward. (R) reverse ^{b}Underlined thymidines (reverse prothrombin primer) were replaced with uracils for the uracil N-glycosylase (UNG) experiment.

All documents mentioned herein are incorporated herein by reference in their entirety.

The following specific references, also incorporated herein by reference, are indicated in the examples and the discussion above by a number in parentheses.

1. Sanger, F., Nicklen, S. & Coulson, A. R. (1977) Proc Natl Acad Sci USA74,5463-7.

2. Cathcart, R. (1990) Nature 347, 310.

3. Prober, J. M., Trainor, G. L., Dam, R. J., Hobbs, F. W., Robertson, C. W., Zagursky, R. J., Cocuzza, A. J., Jensen, M. A. & Baumeister, K. (1987) Science 238, 336-41.

4. Hirsch, M. S., Brun-Vezinet, F., D'Aquila, R. T., Hammer, S. M., Johnson, V. A., Kuritzkes, D. R., Loveday, C., Mellors, J. W., Clotet, B., Conway, B., et al. (2000) Jama 283, 2417-26.

5. Liu, B., Parsons, R. E., Hamilton, S. R., Petersen, G. M., Lynch, H. T., Watson, P., Markowitz, S., Willson, J. K., Green, J., de la Chapelle, A., et al. (1994) Cancer Res 54, 4590-4.

6. Grody, W. W., Cutting, G. R., Klinger, K. W., Richards, C. S., Watson, M. S. & Desnick, R. J. (2001) Genet Med 3, 149-54.

7. Lander, E. S., Linton, L. M., Birren, B., Nusbaum, C., Zody, M. C., Baldwin, J., Devon, K., Dewar, K., Doyle, M., FitzHugh, W., et al. (2001)Nature 409, 860-921.

8. Venter, J. C., Adams, M. D., Myers, E. W., Li, P. W., Mural, R. J., Sutton, G. G., Smith, H. O., Yandell, M., Evans, C. A., Holt, R. A., et al. (2001) Science 291, 1304-51.

9. Bertina, R. M., Koeleman, B. P., Koster, T., Rosendaal, F. R., Dirven, R. J., de Ronde, H., van der Velden, P. A. & Reitsma, P. H. (1994) Nature 369, 64-7.

10. Voorberg, J., Roelse, J., Koopman, R., Buller, H., Berends, F., ten Cate, J. W., Mertens, K. & van Mourik, J. A. (1994) Lancet 343, 1535-6.

11. Poort, S. R., Rosendaal, F. R., Reitsma, P. H. & Bertina, R. M. (1996) Blood 88, 3698-703.

12. Frosst, P., Blom, H. J., Milos, R., Goyette, P., Sheppard, C. A., Matthews, R. G., Boers, G. J., den Heijer, M., Kluijtmans, L. A., van den Heuvel, L. P., et al. (1995) Nat Genet 10,111-3.

13. Seligsohn, U. & Lubetsky, A. (2001) N Engl J Med 344, 1222-31.

14. Wiemann, S., Stegemann, J., Grothues, D., Bosch, A., Estivill, X., Schwager, C., Zimmermann, J., Voss, H. & Ansorge, W. (1995) Anal Biochem 224,117-21.

15. Wiemann, S., Stegemann, J., Zimmermann, J., Voss, H., Benes, V. & Ansorge, W. (1996) Anal Biochem 234, 166-74.

16. Yager, T. D., Baron, L., Batra, R., Bouevitch, A., Chan, D., Chan, K., Darasch, S., Gilchrist, R., lzmailov, A., Lacroix, J. M., et al. (1999) Electrophoresis 20, 1280-300.

17. van den Boom, D., Jurinke, C., Ruppert, A. & Koster, H. (1998) Anal Biochem 256, 127-9.

18. Ruano, G. & Kidd, K. K. (1991) Proc Natl Acad Sci USA 88, 2815-9.

19. Reynolds, T. R., Uliana, S. R., Floeter-Winter, L. M. & Buck, G. A. (1993) Biotechniques 15, 462-4, 466-7.

We claim:

1. A method for substantially simultaneously sequencing multiple nucleic acid targets, comprising:
providing a plurality of nucleic acid targets;
providing a plurality of primers;
annealing of the primers to target sequences of the nucleic acid targets;
sequencing the nucleic acid targets using the primers to obtain a pool of sequence data; and,
analyzing the sequence data without the need to separate the pool of sequence data prior to analysis.

2. The method of claim 1 wherein the pool of sequence data is analyzed substantially simultaneously within a single lane or capillary.

3. The method of claim 1 or 2 wherein the nucleic acid targets are DNA or RNA molecules.

4. The method of claim 3 wherein the nucleic acid targets are single stranded DNA molecules.

5. The method of claim 3 wherein the nucleic acid targets are double stranded DNA molecules.

6. The method of any one of claims 1 through 5 wherein the nucleic acid targets are cDNA, genes or fragments thereof, or non-coding DNA.

7. The method of claim 6 wherein the nucleic acid targets are from the same gene or fragments thereof.

8. The method of claim 6 wherein the DNA nucleic acid targets are from different genes or fragments thereof.

9. The method of any one of claims 1 through 8 wherein the primers are of varying lengths, modifications, and/or size.

10. The method of any one of claims 1 through 9 wherein the primers are modified to comprise abasic regions.

11. The method of any one of claims 1 through 10 wherein the primers are comprised of non-template or template 5′ tails of varying lengths and/or compositions of nucleotides or other molecules.

12. The method of any one of claims 1 through 11 wherein the primers are specific for different target DNA sequences.

13. The method of any one of claims 1 through 11 wherein the primers are specific for the same target DNA sequences.

14. The method of any one of claims 1 through 11 wherein the desired length of the sequence data is varied according to the design of the primer used.

15. The method of claim 14 wherein the shortest desired length of sequence data is at least about one nucleic acid base.

16. The method of any one of claims 1 through 15 wherein the sequencing reaction is uni-directional.

17. The method of any one of claims 1 through 15 wherein the sequencing reaction is bi-directional.

18. The method of any one of claims 1 through 17 wherein the sequencing reaction does not require the separation of the nucleic acids to be separated into different reaction vessels.

19. The method of any one of claims 1 through 18 wherein the nucleic acid targets are pooled from a variety of sources.

20. The method of any one of claims 1 through 19 wherein the method steps are performed in a single reaction vessel.

21. The method of any one of claims 1 through 20 wherein each step of the method is performed in a single reaction vessel.

22. The method of any one of claims 1 through 21 wherein the sequencing reaction of multiple DNA oligonucleotides, or fragments thereof, is performed in a single step without the need to separate each oligonucleotide into separate reaction vessels.

23. The method of any one of claims 1 through 12 wherein the sequence data are analyzed without the need to separate each sequence obtained from said sequencing reaction, before analysis of said data.

24. The method of any one of claims 1 through 23 wherein the plurality of target nucleic acid molecules are amplified by polymerase chain reactions, prior to sequencing.

25. The method of claim 24 wherein the polymerase chain reaction primers are removed from the amplified products prior to sequencing.

26. The method of claim 24 wherein the polymerase chain reaction primers are removed by enzymatic or physical treatment.

27. The method of claim 24 wherein the reverse polymerase chain reaction primers are functionally removed using uracil N-DNA-glycosylase.

28. A method for simultaneously amplifying and sequencing a single nucleic acid molecule or a plurality of nucleic acid molecules, comprising:
providing a single or plurality of target nucleic acid molecules, and a single or a plurality of forward and reverse nucleic acid primer molecules, wherein each primer molecule hybridizes to a distinct area of the target nucleic acid molecules;
amplifying said target nucleic acid molecules; wherein,
deoxyribonucleosides triphosphates are present during the amplifying; wherein,
the number of amplifying cycles are determined by the added concentration of deoxyribonucleosides triphosphates; wherein,
as the amplifying cycles consume the added deoxyribonucleosides triphosphates, the concentrations of free deoxyribonucleosides triphosphates decrease thereby raising the relative concentration of di-deoxyribonucleoside triphosphates.

29. The method of claim 28 wherein deoxyribonucleases triphosphates are added in admixture with the nucleic acid molecules prior to the amplifying.

30. The method of claim 28 or 29 wherein the method steps are performed in a single reaction vessel.

31. The method of claim 28 or 29 wherein each step of the method is performed in a single reaction vessel.

32. The method of any one of claims 28 through 31 wherein a single reaction is provided that comprises the plurality of target nucleic acid molecules and the plurality of forward and reverse nucleic acid primer molecules.

33. The method of any one of claims 28 through 32 wherein varying concentration of deoxyribonucleosides triphosphates are added prior to amplification and the number of amplifying cycles are determined, at least in part, by the added concentration of deoxyribonucleosides triphosphates.

34. The method of any one of claims 28 through 33 wherein a sequencing reaction is favored over amplification as the concentration of di-deoxyribonucleoside triphosphates increase relative to free deoxyribonucleoside triphosphates.

35. The method of any one of claims 28 through 34 wherein the amplifying reaction comprises a polymerase chain reaction.

36. The method of any one of claims 28 through 35 wherein amplification of target nucleic acid molecules via polymerase chain reaction and sequencing of polymerase chain reaction products is performed in a single reaction vessel without the need to process or clean-up the amplified products prior to the sequencing reaction.

37. The method of any one of claims 28 through 36 wherein the concentration of added free deoxyribonucleosides triphosphates determines the number of amplification cycles.

38. The method of any one of claims 28 through 37 wherein the concentration of di-deoxyribonucleosides triphosphates relative to the deoxyribonucleosides triphosphates increases as the deoxyribonucleosides triphosphates are consumed during amplification cycles.

39. The method of any one of claims 28 through 38 wherein the relative free concentrations deoxyribonucleosides triphosphates to di-deoxyribonucleosides triphosphates favors a shift from the amplification reaction to a sequencing reaction.

40. The method of any one of claims 24, 25 or 35 through 39 wherein the polymerase chain reaction is a standard polymerase chain reaction, a ligase chain reaction, reverse transcriptase polymerase chain reaction, Rolling Circle polymerase chain reaction, multiplex polymerase chain reaction, isothermal amplification, strand displacement, and the like.

41. The method of any one of claims 28 through 40 wherein the target nucleic acid molecules are DNA or RNA, and the like.

42. The method of any one of claims 28 through 41 wherein the target nucleic acid molecules are single stranded.

43. The method of any one of claims 28 through 42 wherein the target nucleic acid molecules are double stranded.

44. The method of any one of claims 41 through 43 wherein the target nucleic acid molecules are comprised of eDNA, or genes or fragments thereof, or non-coding nucleic acids or fragments thereof.

45. The method of any one of claims 41 through 44 wherein the target nucleic acid molecules are from the same gene or fragments thereof.

46. The method of any one of claims 41 through 44 wherein the target nucleic acid molecules are from different genes or fragments thereof.

47. The method of any one of claims 28 through 46 wherein the plurality of forward and reverse nucleic acid primer molecules each hybridizes to a distinct area of the target nucleic acid molecules and said primers are of varying lengths, modifications, and sizes.

48. The method of any one of claims 28 through 47 wherein the primers are present at non-equal molar ratios.

49. The method of claim 48 wherein said primers are unmodified, modified, or a combination thereof.

50. The method of any one of claims 28 through 49 wherein one or more of the primers are modified to comprise abasic regions.

51. The method of any one of claims 28 through 50 wherein one or more of the primers comprise non-template or templated 5′ tails of varying lengths.

52. The method of any one of claims 28 through 51 wherein the primers are specific for different target nucleic acid sequences.

53. The method of any one of claims 28 through 51, wherein the primers are specific for the same target nucleic acid sequences.

54. The method of any one of claims 28 through 53 wherein the forward or reverse primer is targeted to a different or same position on the amplified product.

55. The method of claim 54 wherein the modified forward or reverse primers comprises an abasic region.

56. The method of claim 55 wherein the modified reverse primer comprises non-template nucleic acids such as polythymidine tails and is longer in length in relation to the forward primer.

57. The method of claim 55 wherein the modified forward primer comprises non-template nucleic acids such as polythymidine tails and is longer in length in relation to the reverse primer.

58. The method of any one of claims 28 through 57 wherein the forward and reverse primers produce amplified products of varying lengths.

59. The method of any one of claims 28 through 58 wherein the sequencing reaction is uni-directional.

60. The method of any one of claims 28 through 58 wherein the sequencing reaction is bi-directional.

61. The method of any one of claims 28 through 60 wherein the amplification and sequencing reactions do not require the separation of the nucleic acids into different reaction vessels.

62. The method of any one of claims 28 through 60 wherein the amplification and sequencing reactions are performed in a single step.

63. The method of any one of claims 28 through 62 wherein sequencing data obtained from the sequencing reaction is analyzed in a single well on a gel or capillary.

64. The method of claim 63 wherein the sequencing data is analyzed by immobilizing the reverse primer on a solid support.

65. The method of claim 63 wherein the sequencing data is analyzed by using a modified reverse primer such that its migration in the gel or column is slower relative to any other product produced during the amplification and sequencing reactions.

66. The method of claim 65 wherein the reverse primer is modified by biotinylation, blocking group, use of branched primers and the like.

67. The method of any one of claims 1 through 66 wherein the primers are modified by conjugate molecules to further increase the binding affinity and hybridization rate of these oligonucleotides to a target.

68. The method of claim 67 wherein the conjugate molecules are selected from the group consisting of cationic amines, intercalating dyes, antibiotics, proteins, peptide fragments, and metal ion complexes.

69. The method of any one of claims 1 through 68 wherein the primers are modified to increase avidity of binding and/or hybridization rates between a primer and its target nucleic acid.

70. The method of claim 69 wherein the primers are comprised of 2′ modifications to a ribofuranosyl ring of a primer or any other modification.

71. The method of claim 70 wherein said modification comprises a 2′-O-methyl substitution.

72. The method of any one of claims 1 through 71 wherein one or more of the primers are modified to produce varying lengths of amplified and/or sequenced product.

73. The method of any one of claims 1 through 72 wherein one or more of the primers are modified by capping or blocking 3′ ends of primers to prevent or inhibit their use as templates for nucleic acid polymerase activity.

74. The method of claim 73 wherein the primers are capped by addition of 3′ deoxyribonucleotides or 3′, 2′-dideoxynucleotide residues.

75. The method of claim 73 wherein one or more of the primers are capped using non-nucleotide linkers or non-complementary nucleotide residues at the 3′ terminus.

76. The method of claim 75 wherein one or more of the primers have alkane-diol modifications.

77. A method of any one of claims 1 through 76 wherein a disease or disorder is identified.

78. A kit comprising components for performing any of the methods of claims 1 through 77.

79. A kit suitable for substantially simultaneously sequencing multiple oligonucleotides, pooled from a variety of sources, in a single reaction using a single reaction vessel, the kit comprising a plurality of modified primers and a plurality of oligonucleotides.

80. A kit suitable for amplifying and substantially simultaneously sequencing a single nucleic acid molecule or a plurality of nucleic acid molecules in a single reaction within a single reaction vessel, the kit comprising a single or a plurality of target nucleic acid molecule(s), and a single or plurality of forward and reverse nucleic acid primer molecule(s), and reagents for an amplification reaction.

81. The kit of claim 80 comprising deoxyribonucleosides triphosphates.

82. The kit of claim 80 or 81 wherein the one or more of the primers are modified.

83. The kit of any one of claims 80 through 82 wherein the forward primer is targeted to a different position on the amplified product and the reverse primer is of longer length and modified.

84. The kit of any one of claims 77 through 83 wherein the kit comprises a forward or reverse primer that comprises an abasic region.

85. The kit of any one of claims 77 through 84 wherein the kit comprises modified reverse primer that comprise non-template nucleic acids and is longer in length in relation to the forward primer.

86. The kit of claim 85 wherein the one or more modified reverse primers comprise a polythymidine, polycytosine, polyguanine, polyadenine, polyuracil, polyinosine, or other nucleic acid or non-nucleic acid containing tail.

87. The kit of any one of claims 77 through 86 wherein the kit comprises a modified forward primer that comprise non-template nucleic acids and is longer in length in relation to the reverse primer.

88. The kit of any one of claims 77 through 87 wherein the one or more modified forward primers comprise a polythymidine, polycytosine, polyguanine, polyadenine, polyuracil, polyinosine, or other nucleic acid or non-nucleic acid containing tail.

89. Use of a kit of any one of claims 77 through 88 in a diagnostic assay.

90. Use of a method of any one of claims 1 through 89 on a microarray platform.

91. Use of a kit of any one of claims 77 through 88 wherein the primers are used at unequal molar ratios to perform combined amplification and sequencing.

92. Use of a method of any one of claims 1 through 70, wherein the dideoxynucleoside triphosphate is a fluorescently labelled dye terminator or where the primer is fluorescently labelled in the presence of unlabelled ddNTPs.

93. Use of a method of claim 92 wherein the dNTPs or ddNTPs are replaced by their ribonucleotide counterparts.

94. Use of a kit of any one of claims 77 through 88 wherein the dideoxynucleoside triphosphate is a fluorescently labelled dye terminator or where the primer is fluorescently labelled in the presence of unlabelled ddNTPs.

95. Use of a kit of claim 94 wherein the dNTPs or ddNTPs are replaced by their ribonucleotide counterparts
